SEQUENCE LISTING (18 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 238 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTE

SEQ ID NO: 3 - protein, 239 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 4 - protein, 262 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 5 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - protein, 96 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRD

SEQ ID NO: 7 - protein, 10 aa, synthetic construct
VHMPLGFLGP

SEQ ID NO: 8 - protein, 12 aa, synthetic construct
TSTSGRSANPRG

SEQ ID NO: 9 - protein, 16 aa, synthetic construct
EAGRSANHTPAGLTGP

SEQ ID NO: 10 - protein, 5 aa, synthetic construct
GGGGS

SEQ ID NO: 11 - protein, 7 aa, synthetic construct
GGSSGGS

SEQ ID NO: 12 - protein, 10 aa, synthetic construct
GGGGSGGGGS

SEQ ID NO: 13 - protein, 21 aa, synthetic construct
GGGGSVHMGGSGGGGSGGGGS

SEQ ID NO: 14 - protein, 18 aa, synthetic construct
GGGGSGGGGSGGGGSGGS

SEQ ID NO: 15 - protein, 10 aa, synthetic construct
GGGGSVHMGS

SEQ ID NO: 16 - protein, 445 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 215 aa, Homo sapiens
DIQMTQSPSSLSASVGDRVTITCNVDQVIANWLNWYQQKPGKAPKLLIYAASSLQSGVPSRFSGSGSGTDFTLTISSLQPEDFATYYCQQMHPRAPKQFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 215 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQFHKRSPQKFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0254223 A1
(43) Pub. Date: Aug. 1, 2024

(54) RECIPROCALLY MASKED ANTIBODY-CYTOKINE FUSION PROTEINS AND METHODS OF USE THEREOF

(71) Applicant: Novimmune SA, Geneva (CH)

(72) Inventors: Limin SHANG, Bellevue (CH); Giovanni MAGISTRELLI, Cessy (FR); Nicolas FISCHER, Lancy (CH); Elise Sylvie Blanche LECHINE, Ouhans (FR); Pauline MALINGE, Cernex (FR)

(21) Appl. No.: 18/459,562

(22) Filed: Sep. 1, 2023

Related U.S. Application Data
(60) Provisional application No. 63/403,465, filed on Sep. 2, 2022.

(51) Int. Cl. C07K 16/28; A61K 38/00; C07K 14/54; C07K 14/55; C07K 14/715

(52) U.S. Cl. CPC C07K 16/2803; C07K 14/5443; C07K 14/55; C07K 14/7155; A61K 38/00; C07K 2319/30; C07K 2319/50

(57) ABSTRACT

Described herein are compositions and methods to produce masked antibodies useful in a variety of therapeutic indications.

### CROSS-REFERENCE TO RELATED APPLICATIONS

This application claims the benefit of and priority to U.S. Provisional Application No. 63/403,465, filed on Sep. 2, 2022, which is incorporated herein by reference in its entirety.

### FIELD OF THE INVENTION

The present invention relates generally to the composition of masked antibodies and methods of use thereof.

### REFERENCE TO AN ELECTRONIC SEQUENCE LISTING

The contents of the electronic sequence listing (NOVI_051_001US_SeqList_ST26.xml; Size: 18,584 bytes; and Date of Creation: Aug. 28, 2023) are herein incorporated by reference in its entirety.

### BACKGROUND OF THE INVENTION

Antibodies are one of the most successful classes of drugs and benefit from a high target specificity and low intrinsic toxicity. Despite such favorable characteristics, toxicities can arise if the targeted antigen is also expressed at significant levels on non-diseased tissues (Hansel et al. 2010). This is particularly important for the treatment of cancer where the antibodies often mediate cell killing via different mechanisms such as Antibody-Dependent Cellular Cytotoxicity (ADCC), Antibody-Dependent Cellular Phagocytosis (ADCP), Complement Dependent Cytotoxicity (CDC), direct killing of the target cell, Antibody-Drug Conjugates (ADC) or T-cell redirection using bispecific antibodies targeting CD3 on T-cells and a tumor associated antigen on the tumor cells.

Antibodies have been engineered in many ways to improve their efficacy. They have been humanized or isolated from human sequences to decrease immunogenicity potential. Fc domains have been engineered to tune their interaction with Fc receptors and downstream effector function or pharmacokinetic properties. More recently many approaches have been developed to generate bispecific and multispecific antibody formats enabling novel modes of action.

Furthermore, to increase specificity and limit on-target toxicity, novel approaches aim to efficiently activate antibodies when exposed to specific conditions for instance those found within the tumor micro-environment (TME). Diverse engineering strategies have been applied to generate activatable antibodies in the TME or under conditions found in other diseased tissues.

In the last ten years, antibodies have been engineered to become sensitive to a variety of stimuli including pH, light, temperature, ions, effector molecules, antigen combinations and proteases (Lucchi et al. 2021). As such, under specific conditions these antibodies become activated, i.e., can bind their antigen, or not. One of the main approaches exploited to activate antibodies is to rely on specific preferentially proteases expressed in the TME. Protease-activated antibodies are based on the introduction of masking domains that block the antibody binding to its cognate antigen and that are connected via a cleavable linker. Once the antibody reaches the tumor site, the cleavable linkers are cleaved by the proteases, releasing the masking domains, and restoring antibody binding activity at tumor site. As cleavage is mediated by proteases overexpressed in the TME, in contrast, in healthy tissue with low protease activity, the interaction between the antibody and its target is prevented by the masking domain, limiting on-target off-tumor toxicity.

Different masking strategies can be employed to hinder paratope-epitope interaction. Affinity-based masks are specific for a given antibody and occupy the antibody paratope so that it is not able to interact with the epitope on the antigen. Generally, the interaction must be of weak or intermediate affinity so that upon cleavage of the linker, the mask gets released from the antibody. Thus, the affinity of the mask is an important parameter to adjust for each antibody-mask pair. Affinity masks can be peptides of anti-idiotypic antibody fragments. On the other hand, steric hindrance-based masks do not interact specifically with the antibody paratope but inhibit antibody binding through steric hindrance (Bleuez et al. 2022).

Affinity-based masks were first introduced in 2009. Recombinant Epidermal Growth Factor Receptor (EGFR) fragments were fused to single-chain Fv fragments (scFv) derived from antibodies targeting EGFR via a linker cleavable by proteases expressed in the TME. Masked scFv is poorly associated with EGFR, whereas protease treatment providing unmasked scFv restored association. Since this first example, various affinity-based masked antibodies have emerged. Amongst them, anti-CD166 (CX-2009), anti-PD-L1 (CX-072), and anti-CD71 (CX-2029) antibodies have reached clinical trials. An anti-CTLA-4 (XT101) demonstrated tumor-selective pharmacodynamic effects and efficacy in preclinical models. For the design of affinity-based masks, the selected peptide must have an appropriate affinity to the paratope of the antibody to mask its binding but also have a weak enough affinity to be released once cleaved. Moreover, for each antibody, a specific peptide must be developed.

Steric hindrance mask was introduced more recently, in 2017, with Chen et al. They demonstrated that fusing Latency-Associated Peptide (LAP) to an anti-EGFR or an anti-TNFα antibody could reduce their binding activity that could be restored after protease cleavage. Similarly, addition of polyethylene glycol (PEG) chains to recombinant proteins as well as antibody fragment has been demonstrated to hinder protein-protein interactions and biological activity via steric hindrance of the large PEG chain masking non-specifically the protein-protein interaction surfaces.

In healthy tissues, extracellular protease levels are usually low and their activity is tightly regulated by inhibitors present in the tissue. Whereas in tumor tissue their expression levels and activity can be significantly upregulated. Altered proteases expression and activity are a hallmark of cancer, which play an important role in cancer development at multiple stages from tumor formation to metastasis (Vasiljeva et al. 2019). For example, proteases are implicated in cancer cell invasion in healthy tissues by the degradation of basement membranes and extracellular matrix (ECM).

Matrix metalloproteinases (MMPs) and urokinase-type Plasminogen Activator (uPA) are among the upregulated proteases inside the TME. MMPs are a family of zinc-endopeptidases and are implicated in cancer development, progression, and angiogenesis. They are upregulated in many cancer types. 23 MMPs have been identified in human, amongst them, MMP-9 is implicated in many cancer development processes. uPA is a serine-endopeptidase involved in the regulation of tumor progression and metastasis. More specifically, uPA cleaves plasminogen leading to active plasmin which initiates the degradation of the components of the ECM (Mahmood et al. 2011). MMP9 and uPA are often exploited in the design of activatable antibodies.

Cytokines are key players of immune responses and mediate cell-to-cell communication, making them interesting therapeutic agents (Berraondo et al, 2019). Interleukins, such as IL-2, IL-6, IL-7, IL-12, IL-15 and IL-21 can be used for the treatment of cancers and other diseases. However, therapeutic usage via systemic administration often leads to undesired side effects, such as low blood pressure, flu-like symptoms, nausea, diarrhea and arrhythmia.

Amongst them, interleukin-2 (IL-2) and interleukin-15 (IL-15) are related cytokines able to stimulate immune cells via interactions with their receptors. They bind to their respective alpha receptor subunit (IL-2Rα and IL-15Rα) and with their shared receptors beta and gamma subunits (IL-2/IL-15Rβγ). IL-2 is known as mediator of expansion, differentiation, and survival of T cells. While IL-15 is known as mediator of expansion and differentiation of NK and CD8 memory T cells.

IL-2 received the FDA approval for the treatment of advanced and metastatic melanoma. Nevertheless, systemic administration of IL-2 is associated with severe side effects, limiting its clinical use. Moreover, IL-2 is involved in the development of regulatory T cells (Tregs) that prevents the development of effective antitumor immunity.

Therefore, it remains a need to develop cytokine therapeutics that mediate immune activation specifically at the tumor site without the systemic side effects. One way to limit side effects of IL-2 would be to make it specifically active at tumor site (Puskas et al., 2011). Moreover, combining IL-2 with extracellular domain (ECD) of IL-2Rα could preferentially activate IL-2Rβ and γ expressed by CD8 T cells and NK cells but limiting the activation of Tregs expressing IL-2Rα.

The combination of cytokines and their receptors as steric hindrance masks in activatable antibodies would not only maintain the cytokine inactive but also the linked antibody inactive outside the tumor. Upon reaching the TME, cleavage of the linkers by proteases over-expressed in TME would release both the active cytokine and the functional antibody. This strategy would allow a double therapeutic effect: activation of both antibody and cytokine at the tumor site while limiting undesired activation in healthy tissues and systemic circulation.

### SUMMARY OF THE INVENTION

In various aspects the invention provides an antibody fusion protein having the following structure:

A first antigen binding domain having a first heavy chain polypeptide (H1) and a first light chain polypeptide (L1); and a second antigen binding domain comprising a second heavy chain polypeptide (H2) and a second light chain polypeptide (L2). A cytokine is linked via a first protease cleavable linker: (i) to a N-terminus of the L1 and/or the L2; (ii) to the N-terminus of the H1 and/or H2; or (iii) to a N-terminus of the L1, L2, H1 and/or H2.

The antibody fusion protein described above, that further has at least a portion of the cognate receptor of the cytokine linked via a second protease linker to the N-terminus of the H1 and/or H2 or to the N-terminus of the L1 and/or L2. Different cytokines can be incorporated in the constructs of the invention, such as IL-2 or IL-15. The portion of the cytokine receptor can be an extracellular portion of their respective cognate receptors such as IL-2Ra, IL-2RB or IL-2Ry, for IL-2 or combinations thereof.

The antibody fusion proteins described above, that further have the cytokine and its cognate receptors being sequentially linked to the N-terminus of the same light or heavy chain of an antibody in different orders using identical or different linkers.

The components of the invention can be combined in different ways to achieve varying levels of masking of i) the antigen binding domain and ii) the cytokine, depending on the anticipated mode of action of the fusion protein as well as the potential toxicity of the unmasked antigen binding domain or the cytokine.

In some aspects of the invention, the cytokine sequence can be modified to alter its interaction with various receptors and thus modulate its biological activity when masked or unmasked. Similarly, the receptor sequence can also be modified to alter its interaction with the cognate cytokine. In addition, the affinity or the antigen binding domain can be modified to adjust its binding capacity when masked or unmasked.

If the antigen binding domain contains an Fc domain (for instance in an antibody), the Fc can be selected for its capacity to engage Fc receptors and drive effector functions such as ADCC, or CDC. The Fc portion can also be silenced or enhanced by introduction of mutations to further modulate the activity.

### BRIEF DESCRIPTION OF THE DRAWINGS

FIG. 1 is an illustration of the reciprocal masking and activation approach by fusing a cytokine receptor complex to the N-termini of an antibody. The masking can be removed by proteolytic cleavage. The antibody has no binding specificity for the cytokine or cytokine receptor complex.

FIG. 2 shows different possible configurations of antibody-cytokine/receptor fusions.

FIG. 3 illustrates different constructs generated. The “n°” notation represents different Novimmune construct configurations.

FIG. 4 depicts strategies and combinations of elements to achieve a desired mode of action.

FIG. 5 is an illustration of the reciprocal masking and activation approach using a CD47 antibody and IL2-IL2Rα as an example. In this case, unmasking of the antibody-cytokine/receptor fusion in the tumor microenvironment restores CD47-SIRPα blockade, leading to increased phagocytic activity of tumor cells as well as IL-2 signaling mediating T cell activation and proliferation.

FIG. 6 is an example of vector maps generated for the expression of different construct configurations, that are represented schematically.

FIG. 7A, FIG. 7B, FIG. 7C, FIG. 7D, FIG. 7E, and 7F show SDS-PAGE analyses of antibody-cytokine/receptor fusions before and after proteolytic cleavage by MMP-9.

FIG. 8 shows an example of the binding profiles of an antibody-cytokine/receptor fusion before and after cleavage using Bio-Layer Interferometry (BLI) technology.

FIG. 9A, FIG. 9B, FIG. 9C, FIG. 9D, FIG. 9E, FIG. 9F, FIG. 9G, FIG. 9H, FIG. 9I, FIG. 9J, FIG. 9K, FIG. 9L, FIG. 9M, FIG. 9N, FIG. 9O, FIG. 9P, FIG. 9Q, FIG. 9R, FIG. 9S, FIG. 9T, FIG. 9U, FIG. 9V, FIG. 9W, FIG. 9X, FIG. 9Y, FIG. 9Z, and FIG. 9AB show the binding profiles on CD47 at the surface of Peak cells of antibody-cytokine/receptor fusions before and after proteolytic cleavage by MMP-9.

FIG. 10A, FIG. 10B, FIG. 10C, FIG. 10D, FIG. 10E, FIG. 10F, FIG. 10G, FIG. 10H, FIG. 10I, FIG. 10J, FIG. 10K, FIG. 10L, FIG. 10M, FIG. 10N, FIG. 10O, FIG. 10P, FIG. 10Q, FIG. 10R, FIG. 10S, FIG. 10T, FIG. 10U, FIG. 10V, FIG. 10W, FIG. 10X, FIG. 10Y, and FIG. 10Z show the IL-2 signaling activity of antibody-cytokine/receptor fusions before and after proteolytic cleavage by MMP-9 using the HEK-Blue™ IL-2 reporter system.

FIG. 11A, FIG. 11B, FIG. 11C, FIG. 11D, FIG. 11E, FIG. 11F, FIG. 11G, FIG. 11H, FIG. 11I, FIG. 11J, FIG. 11K, FIG. 11L, and FIG. 11M show the IL-2 signaling activity of antibody-cytokine/receptor fusions before and after proteolytic cleavage by MMP-9 using the HEK-Blue™ CD122/CD132 reporter system.

### DETAILED DESCRIPTION OF THE INVENTION

The present disclosure provided for the generation of protease activatable antibodies and cytokines or cytokine/receptor fusions within a single construct. The cytokine and/or the cytokine/receptor is masking the antibody combining site and conversely the antibody masking the cytokine or cytokine/receptor. The reciprocal masking reduces simultaneously the biological activity of the antibody and the cytokine or cytokine/receptor. The reciprocal masking activity being mediated by steric hindrance as the antibody has no affinity for the cytokine/receptor. The antibody is linked to the cytokine and or cytokine/receptor via one or more protease cleavable linkers. Upon cleavage by proteases that are upregulated in the TME, both the antibody and the cytokine/receptor are released into the TME in a form that has fully or partially restored biological activity.

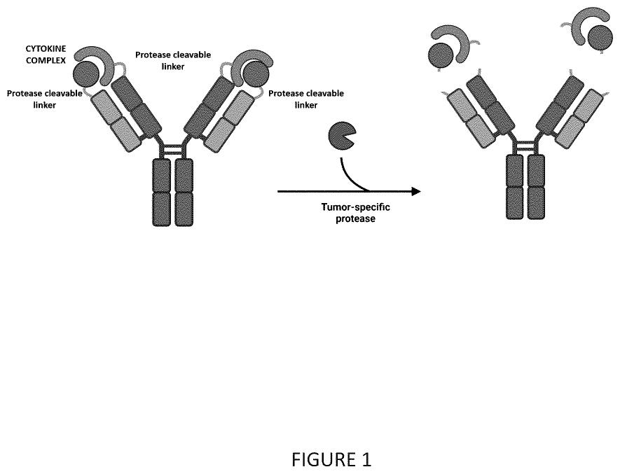

Accordingly, the present disclosure allows for: (i) the reciprocal double masking of the antibody-cytokine/receptor fusion in the circulation and within healthy tissues; (ii) the release upon proteolytic cleavage of active molecules (i.e., antibody, cytokine, cytokine receptor); (iii) the unmasked antibody that can engage its target (i.e., binds specifically to its cognate antigen); (iv) the biologically active cytokine/receptor that can signal via its cognate receptor; (v) an increased therapeutic activity of the antibody-cytokine/receptor cytokine fusion is obtained as two different modalities are released in comparison to previous masking strategies in which the mask has no function upon release. (See FIG. 1)

Different molecular designs and structures can be used to generate antibody-cytokine/receptor fusions of the invention.

In a first configuration the cytokine is fused to the N-terminus of the antibody light chain and an extracellular portion of the cytokine receptor is fused to the N-terminus of the antibody heavy chain. This configuration allows for the cytokine to interact with the extracellular portion of the cytokine receptor. As the N-termini of the heavy and light chains are close to the antibody combining site, steric hindrance mediated by the fusion of cytokine/receptor as well as reciprocal inhibition of cytokine activity are facilitated by this configuration.

In a second configuration the cytokine is fused to the N-terminus of the antibody heavy chain and an extracellular portion of the cytokine receptor is fused to the N-terminus of the antibody light chain. In this swapped configuration, the steric hindrance is also facilitated as described in the first configuration.

In other configurations, only the cytokine is fused to either the N-terminus of the light chain or the N-terminus of the heavy chain.

In other configurations, the cytokine is fused to both the N-terminus of the light chain and the N-terminus of the heavy chain.

In other configurations, the cytokine and a first extracellular portion of the cytokine receptor are fused to the N-terminus of the light chain and a second extracellular portion of the cytokine receptor is fused to the N-terminus of the antibody heavy chain. Such configurations can increase the masking of the cytokine.

In other configurations, the cytokine and a first extracellular portion of the cytokine receptor are fused to the N-terminus of the heavy chain and a second extracellular portion of the cytokine receptor is fused to the N-terminus of the antibody light chain. Similarly, such configurations can increase the masking of the cytokine.

In other configurations, the cytokine is fused to the N-terminus of the light chain and two extracellular portions, that is the first extracellular portion and the second extracellular portion of the cytokine receptor are fused to the N-terminus of the antibody heavy chain to further block the cytokine activity.

In other configurations, the cytokine is fused to the N-terminus of the heavy chain and two extracellular portions, that is the first extracellular portion, and the second extracellular portion of the cytokine receptor are fused to the N-terminus of the antibody light chain to further block the cytokine activity.

In each of the possible configurations described above, the linkers and protease cleavable sequences can be varied to simultaneously optimize both masking and proteolytic cleavage efficiency. In some embodiments, the antibody fusion protein is comprised of a first protease cleavable linker. In some embodiments, the antibody fusion protein is comprised of a first protease cleavable linker and a second protease cleavable linker. In some embodiments, the antibody fusion protein is comprised of a first protease cleavable linker, a second protease cleavable linker, and a third protease cleavable linker. In some embodiments, the antibody fusion protein is comprised of three or more cleavable linkers. In some embodiments, the cleavable linker is one or more of SEQ ID NO: 7 (CM1), SEQ ID NO: 8 (CM2), or SEQ ID NO:9 (CM3).

In some embodiments, the cleavable linker is cleaved by a protease or peptidase that is upregulated or present at higher amounts in the TME compared to healthy peripheral tissues. In some embodiments, the cleavable linker is cleaved by MMP-9.

In each of the possible configurations described above, the cytokine can be modified by mutagenesis to alter its interaction with one or several of its cognate receptors and modify its biological activity.

In some embodiments, the antibody fusion protein comprises a first portion of the cognate receptor of the cytokine. In some embodiments, the antibody fusion protein comprises a second portion of the cognate receptor of the cytokine. In some embodiments, the first portion of the cognate receptor is any one of the extracellular portions of IL-2Ra, IL-2RB, IL-2Ry, IL-15Ra Sushi 1. In some embodiments, the second portion of the cognate receptor is any one of the extracellular portions of IL-2Ra, IL-2RB, IL-2Ry, IL-15Ra Sushi 1. In some embodiments, the first portion of the cognate receptor is IL-2Ra and the second portion of the cognate receptor is IL-2RB. In some embodiments, the first portion of the cognate receptor is IL-2RB and the second portion of the cognate receptor is IL-2Ra. In some embodiments, the first portion of the cognate receptor is IL-2Ra and the second portion of the cognate receptor is IL-2Ry. In some embodiments, the first portion of the cognate receptor is IL-2Ry and the second portion of the cognate receptor is IL-2Ra.

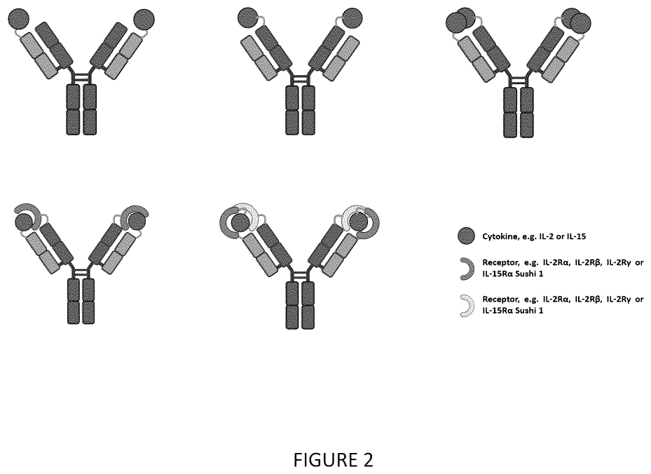

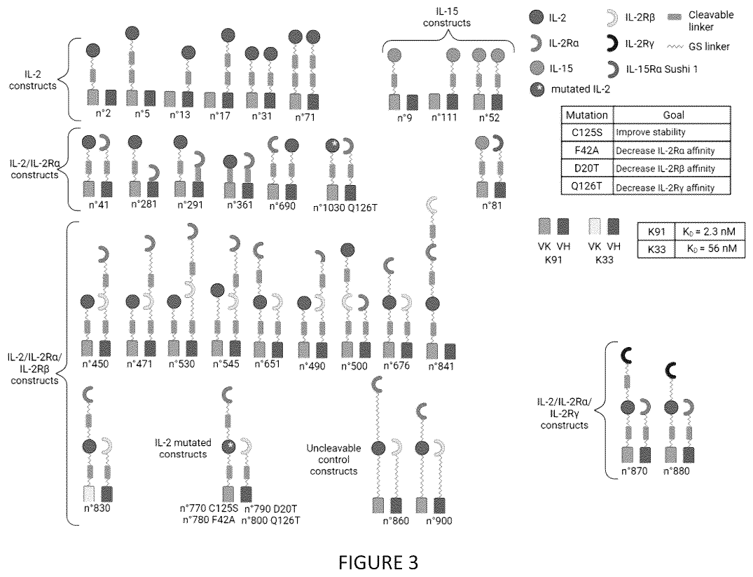

Other configurations combining a cytokine and one or several extracellular portions of its receptor can be linked to the N-termini of the heavy and/or light chain of an antibody using protease cleavable or non-cleavable linkers to achieve various degree of masking of the cytokine and the antibody. A non-exhaustive representation of possible configurations is shown in FIG. 2 and FIG. 3.

In some of the constructs, the cytokine used for N-terminal fusion can be but not limited to IL-2, IL-4, IL-7, IL-9, IL-15, IL-21 and the domain receptor taken from their respective receptors, including IL-2Rα or IL-2Rβ or IL-2Rγ or any combination thereof if the cytokine used is IL-2. In some embodiments, the mutated cytokine is a mutated IL-2 or a mutated IL-15. In some embodiments, the mutated IL-2 comprises one or more of a C125S mutation, a F42A mutation, a D20T mutation, or a Q126T mutation.

Any antibody can be masked according to the invention and can be for example, but not limited to antibodies targeting CD47, CD3, CD28, PD-1, PD-L1, PD-L2, CTLA-4, 4-1BB, CD40, CD40L, OX40, OX40L, ICOS, ICOSL, CD70, CD27, CD28, GITR, GITRL, TIGIT, TIM3, LAG3, CEACAM5, EGFR, SIRPα, CD20, CD19, BCMA, FcRH5, CD38, PSMA, CD73, HER2, HER3, cMet, GPC3, EpCAM, GPRC5D, MUC-16. In some embodiments, the antibody fusion protein comprises an antibody specific for CD47 such as K91 antibody or K33 antibody.

Different sequences can be used as linker and protease sensitive linkers to connect the antibody and the cytokine or cytokine receptor domain.

The affinity of the antibody or antigen binding component can be modified to optimize the difference in biological activity between the masked and unmasked forms so that potential peripheral toxicities can be minimized while anti-tumor activity is maintained.

Similarly, the activity of the cytokine can be modified to optimize the difference in biological activity between the masked and unmasked forms so that potential peripheral toxicities can be minimized while anti-tumor activity is maintained.

The antibody Fc domain can be selected for its capacity to engage Fc receptors and drive effector functions such as ADCC, ADCP or CDC. The Fc portion can also be silenced or enhanced by introducing mutations to further modulate the activity. The choice of Fc in combination with different configurations can lead to antibody-cytokine receptors with different safety and activity profiles that can be exploited in the context of the present invention.

The different components described above, i.e., location of cytokine fusion, presence and number of extracellular portions of the cytokine receptor, affinity of the antibody, modification of cytokine activity as well as choice of the Fc portion, can be combined to find the optimal configuration depending on the antibody and cytokine used, to obtain the desired mode of action and optimal safety and efficacy. Mutations that enhance or reduce Fc gamma receptor or complement interaction can be pursued herein. Mutations that modulate FcRn interaction to alter antibody half-life can be pursued herein. A list of possible mutations is described in Antibodies 2020, 9(4), 64; https://doi.org/10.3390/antib9040064, which is incorporated herein in its entirety.

In some embodiments, the Fc comprises at least one L234A, or a L235A, or a P329A mutation. In some embodiments, the Fc comprises a L234A, a L235A, and a P329A mutation.

In ideal scenarios, the construct is effective at blocking both the cytokine and the antibody and thus can incorporate a high affinity antibody and a cytokine retaining full activity. High affinity antibodies and fully active cytokines can also be used if the antibody has limited toxicity in the periphery and cytokine blockade is effective.

If this cannot be achieved or if the desired mode of action is more antibody-driven, the focus can be put on efficient antibody masking enabling the use of a potent antibody with high toxicity or other liabilities in the periphery. In that case, a lower potency cytokine can deliberately be incorporated in the fusion construct if optimal cytokine masking is difficult to achieve in the context of optimal antibody blockade.

Conversely, if cytokine function is the main driver of the intended mode of action, the focus can be placed on effective cytokine blockade and incorporating a lower affinity antibody thus limiting its unwanted effects in the periphery.

Furthermore, the choice of an active or less active or inactive Fc portion brings another layer of optimizing activity in the tumor and limiting peripheral toxicities. For instance, if the focus of the construct is on the cytokine component and that the antibody is not fully blocked, a high affinity antibody can still be used if the Fc is silent to limit off-tumor side effects.

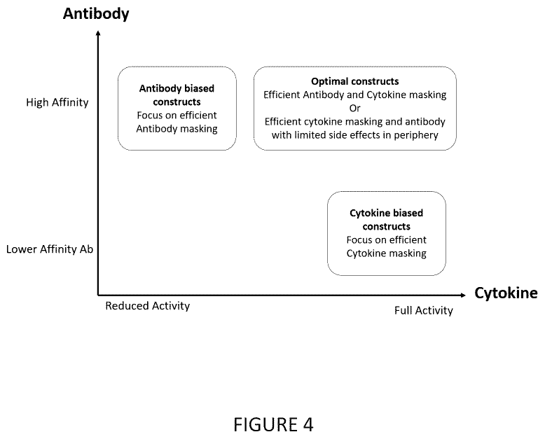

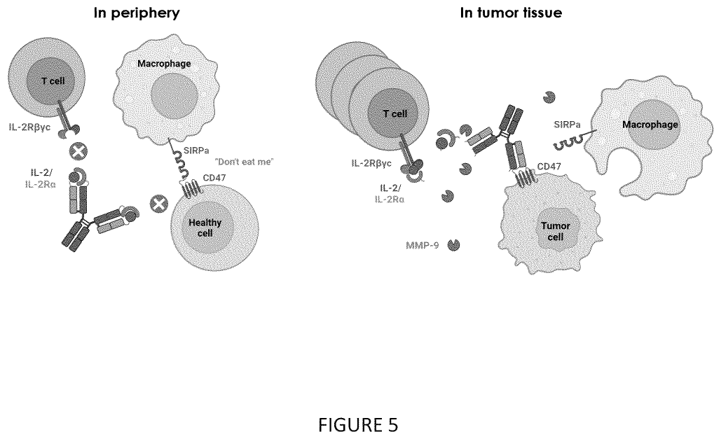

The combinations of elements and strategies to achieve a desired mode of action that are enabled by the invention are depicted in FIG. 4.

In one embodiment, the antibody used is a high affinity anti-human CD47 antibody and the cytokine receptor complex is IL-2 and IL-2Rα and/or IL-2Rβ and/or IL-2Rγ. CD47 is overexpressed in a wide range of cancers but is also ubiquitously expressed in healthy tissues including red blood cells (RBCs). Interaction of CD47 to transmembrane signal-regulatory protein-α (SIRPα) expressed at the surface of macrophages inhibits phagocytosis. Thus, by blocking the interaction of CD47 with SIRPα, phagocytes are activated and can mediate phagocytosis. However, an anti-CD47 antibody will bind and block CD47 on every cell leading to unwanted toxicities and poor pharmacokinetic properties that were observed with anti-CD47 monoclonal antibodies administered to patients. Thus, according to the invention, a CD47 antibody masked with a cytokine/receptor complex would not bind effectively CD47 in the periphery limiting toxicities and improving pharmacokinetic properties and, upon activation by proteolytic cleavage in the TME, could block CD47-SIRPα interaction effectively. This blockade enhances activity of phagocytes and activates the innate immune system. Simultaneously the released IL-2 can activate immune cells including T-cells within the TME while avoiding toxicities in the periphery. The reciprocal masking and activation approach using a CD47 antibody and IL-2/IL-2Rα as an example is illustrated in FIG. 5.

The components of the invention are not limited to monoclonal antibodies of any isotype or containing mutations modulating Fc mediated activities, but are also applicable to other antibody formats including, but not limited to, antibody fragments, bispecific antibodies, antibody drug conjugates, antibody fusion proteins and other binding protein scaffolds such as single domain antibodies (e.g camelid VHHs).

In some embodiments, the antibody fusion protein is a human IgG1, or a human IgG2, or a human IgG3, or a human IgG4, or a human IgA, or a human IgE, or a human IgM.

In some embodiments, the antibody fusion protein is a bispecific antibody, wherein the first antigen binding domain binds to a first antigen and the second antigen binding domain binds to a second antigen, wherein the first antigen and the second antigen are not the same antigen.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

### Example 1. Design and Molecular Cloning of Antibody-Cytokine/Receptor Fusions

The anti-CD47 antibodies K91 and K33 (See, U.S. Ser. No. 17/701,573 (NOVI-048/001US, the contents of which is hereby incorporated by reference in its entirety) were used for the design of several antibody-cytokine/receptor fusions (See, Table 1). Either IL-2 (SEQ ID NO: 1) or IL-15 (SEQ ID NO: 5) or IL-2Rα (SEQ ID NO: 2) or IL-2Rβ (SEQ ID NO: 3) and/or IL-2Rγ (SEQ ID NO: 4), or the sushi domains of IL-15Rα (SEQ ID NO: 6), were fused at the N-terminus of either the antibody light chain or heavy chain using different sets of connecting linkers (Linker 1, 2, 3, 4, 5 and 6) as well as linkers that can be cleaved by selected proteases. Cleavable moiety 1 (CM1) referred to the linker sequence cleavable by the tumor protease MMP-9 (VHMPLGFLGP; SEQ ID NO:7), cleavable moiety 2 (CM2) referred to the linker cleavable by uPA (TSTSGRSANPRG; SEQ ID: NO:8) and cleavable moiety 3 (CM3) referred to the linker cleavable by uPA, matriptase, legumain, MMP-2/7/9/14 (EAGRSANHTPAGLTGP; SEQ ID NO:9). Cleavable linkers were preceded and followed by a flexible Glycine Serine (GS) linker kept short to favorize steric hindrance. In some of the constructs containing IL-2 as a cytokine component, mutations were introduced into the IL-2 sequence to either stabilize IL-2 or modify its interaction with different components of the IL-2R. Some control constructs that did not contain cleavable linker (n860 and n900) were also designed.

Different constructs generated are described in Table 1 and illustrated in FIG. 3.

TABLE 1
Structure and composition of masked antibody constructs. Detailed structures of
masked antibodies with single or multiple masking domains with different components described,
the constructs are numbered to facilitate comprehension.

| | | | | | Linker | | Structure |
|---|---|---|---|---|---|---|---|
| Construct | | Masking | Linker | Cleavable | (L2/L4/ | | (N- to C- |
| No | Name | domain | (L1/L3/L5) | linker | L6) | Domain | terminal) |
| 46 | pNOVI V2K K91 | — | — | — | — | VKCK | VKCK |
| | | — | — | — | — | VHCH | VHCH |
| 22 | pNOVI V2K K33 | — | — | — | — | VKCK | VKCK |
| | | — | — | — | — | VHCH | VHCH |
| 2 | pNOVI V2K K91_IL- | IL-2 (1-153) | GGGGS (SEQ ID | CM1 | GGS | VKCK | IL-2-L1- |
| | 2 CM1_VKCK | | NO: 10) | | | | CM1-L2- |
| | | | | | | | VKCK |
| | | | — | — | — | VHCH | VHCH |
| 5 | pNOVI V2K K91_IL- | IL-2 (1-153) | GGSSGGS (SEQ | CM1 | GGS | VKCK | IL-2-L1- |
| | 2 CM1_CM2_VKCK | | ID NO: 11) | GG | | | CM1-CM2- |
| | | | | CM2 | | | L2-VKCK |
| | | — | — | — | — | VHCH | VHCH |
| 13 | pNOVI V2K K91_IL- | — | — | — | — | VKCK | VKCK |
| | 2 CM1_VHCH | IL-2 (1-153) | GGSSGGS (SEQ | CM1 | GGS | VHCH | IL-2-L1- |
| | | | ID NO: 11) | | | | CM1-L2- |
| | | | | | | | VHCH |
| 17 | pNOVI V2K K91_IL- | — | — | — | — | VKCK | VKCK |
| | 2 CM1_CM2_VHCH | IL-2 (1-153) | GGSSGGS (SEQ | CM1 | GGS | VHCH | IL-2-L1- |
| | | | ID NO: 11) | GG | | | CM1-CM2- |
| | | | | CM2 | | | L2-VHCH |
| 31 | pNOVI V2K K91_IL- | IL-2 (1-153) | GGGGS (SEQ ID | CM1 | GGS | VKCK | IL-2-L1- |
| | 2 CM1_VKCK_IL- | | NO: 10) | | | | CM1-L2- |
| | 2_CM1_VHCH | | | | | | VKCK |
| | | IL-2 (1-153) | GGSSGGS (SEQ | CM1 | GGS | VHCH | IL-2-L1- |
| | | | ID NO: 11) | | | | CM1-L2- |
| | | | | | | | VHCH |
| 41 | pNOVI V2K K91_IL- | IL-2 (1-153) | GGGGS (SEQ ID | CM1 | GGS | VKCK | IL-2-L1- |
| | 2 CM1_VKCK_IL- | | NO: 10) | | | | CM1-L2- |
| | | | | | | | VKCK |
| | 2Rα_CM1_VHCH | IL-2Rα (1-238) | GGGGS (SEQ ID | CM1 | GGS | VHCH | IL-2Rα-L1- |
| | | | NO: 10) | | | | CM1-L2- |
| | | | | | | | VHCH |
| 71 | pNOVI V2K K91_IL- | IL-2 (1-153) | GGSSGGS (SEQ | CM1 | GGS | VKCK | IL-2-L1- |
| | 2_CM1_CM2_VKCK_IL- | | ID NO: 11) | GG | | | CM1-CM2- |
| | 2_CM1_CM2_VHCH | | | CM2 | | | L2-VKCK |
| | | IL-2 (1-153) | GGSSGGS (SEQ | CM1 | GGS | VHCH | IL-2-L1- |
| | | | ID NO: 11) | GG | | | CM1-CM2- |
| | | | | CM2 | | | L2-VHCH |
| 9 | pNOVI V2K | IL-15 (1-162) | GGGGS (SEQ ID | CM1 | GGS | VKCK | IL-15-L1- |
| | K91_IL15_CM1_VKCK | | NO: 10) | | | | CM1-L2- |
| | | | | | | | VKCK |
| | | — | — | — | — | VHCH | VHCH |
| 111 | pNOVI V2K | — | — | — | — | VKCK | VKCK |
| | K91_IL15_CM1_VHCH | IL-15 (1-162) | GGSSGGS (SEQ | CM1 | GGS | VHCH | IL-15-L1- |
| | | | ID NO: 11) | | | | CM1-L2- |
| | | | | | | | VHCH |
| 52 | pNOVI V2K | IL-15 (1-162) | GGGGS (SEQ ID | CM1 | GGS | VKCK | IL-15-L1- |
| | K91_IL15_CM1_VKCK_ | | NO: 10) | | | | CM1-L2- |
| | IL15_CM1_VHCH | | | | | | VKCK |
| | | IL-15 (1-162) | GGSSGGS (SEQ | CM1 | GGS | VHCH | IL-15-L1- |
| | | | ID NO: 11) | | | | CM1-L2- |
| | | | | | | | VHCH |
| 81 | pNOVI V2K | IL-15 (1-162) | GGGGS (SEQ ID | CM1 | GGS | VKCK | IL-15-L1- |
| | K91_IL15_CM1_VKCK_ | | NO: 10) | | | | CM1-L2- |
| | IL15Rα_Sushi 1_ | | | | | | VKCK |
| | CM1_VHCH | IL-15Rα | GGGGS (SEQ ID | CM1 | GGS | VHCH | IL-15Rα |
| | | Sushi 1 (1-96) | NO: 10) | | | | Sushi 1- |
| | | | | | | | L1-CM1- |
| | | | | | | | L2-VHCH |
| 281 | pNOVI V2K K91_IL- | IL-2 (1-153) | GGGGS (SEQ ID | CM1 | GGS | VKCK | IL-2-L1- |
| | 2_CM1_VKCK_IL- | | NO: 10) | | | | CM1-L2- |
| | 2Rα WO LIN_VHCH | | | | | | VKCK |
| | | IL-2Rα (1-238) | — | — | — | VHCH | IL-2Rα- |
| | | | | | | | VHCH |
| 291 | pNOVI V2K K91_IL- | IL-2 (1-153) | GGGGS (SEQ ID | CM1 | GGS | VKCK | IL-2-L1- |
| | 2_CM1_VKCK_IL- | | NO: 10) | | | | CM1-L2- |
| | 2Rα WO LIN | | | | | | VKCK |
| | CM1_VHCH | IL-2Rα (1-238) | — | CM1 | — | VHCH | IL-2Rα- |
| | | | | | | | CM1- |
| | | | | | | | VHCH |
| 361 | pNOVI V2K K91_IL- | IL-2 (1-153) | — | CM1 | — | VKCK | IL-2-CM1- |
| | 2 WO LIN CM1 | | | | | | VKCK |
| | VK_IL-2Rα WO LIN | IL-2Rα (1-238) | — | CM1 | — | VHCH | IL-2Rα- |
| | CM1_VH | | | | | | CM1- |
| | | | | | | | VHCH |
| 450 | pNOVI V2K | IL-2 (1-153) | GGGGS (SEQ ID | CM1 | GGS | VKCK | IL-2-L1- |
| | K91_A_IL- | | NO: 10) | | | | CM1-L2- |
| | 2_1_153_CM1 | | | | | | VKCK |
| | VKCK_IL- | IL-2Rα (1-238) | GGGGS (SEQ ID | CM1 | GGS | VHCH | IL-2Rα-L1- |
| | 2R_1_238_CM1_IL- | | NO: 10) | | | | CM1-L2-IL- |
| | 2Rβ_27_239_CM1 | IL-2Rβ (27- | GGGGS (SEQ ID | CM1 | GGS | | 2Rβ-L3- |
| | VHCH | 239) | NO: 10) | | | | CM1-L4- |
| 471 | pNOVI V2K | IL-2 (1-153) | GGGGS (SEQ ID | CM1 | GGS | VKCK | IL-2-L1- |
| | K91_A1_IL- | | NO: 10) | | | | CM1-L2- |
| | 2_1_153_CM1 | | | | | | VKCK |
| | VKCK IL- | IL-2Rα (1-238) | (GGGGS)2 (SEQ | CM1 | GGS | VHCH | IL-2Rα-L1- |
| | 2Rα_1_238_CM1_IL- | | ID NO: 12) | | | | CM1-L2-IL- |
| | 2Rβ_27_239_CM1 | IL-2Rβ (27-239) | GGGGS (SEQ ID | CM1 | GGS | | 2Rβ-L3- |
| | VHCH | | NO: 10) | | | | CM1-L4- |
| | | | | | | | VHCH |
| 530 | pNOVI V2K | IL-2 (1-153) | GGGGS (SEQ ID | CM1 | GGS | VKCK | IL-2-L1- |
| | K91_A2_IL- | | NO: 10) | | | | CM1-L2- |
| | 2_1_153_CM1 | | | | | | VKCK |
| | VKCK IL- | IL-2Rα (1-238) | (GGGGS)2 (SEQ | CM1 | GGS | VHCH | IL-2Rα-L1- |
| | 2Rα_1_238_CM1_IL- | | ID NO: 12) | | | | CM1-L2-IL- |
| | 2Rβ_27_239_CM1 | IL-2Rβ (27-239) | (GGGGS)2 (SEQ | CM1 | GGS | | 2Rß-L3- |
| | VHCH | | ID NO: 12) | | | | CM1-L4- |
| | | | | | | | VHCH |
| 545 | pNOVI V2K | IL-2 (1-153) | (GGGGS)2 (SEQ | CM1 | GGS | VKCK | IL-2-L1- |
| | K91_A3_IL- | | ID NO: 12) | | | | CM1-L2- |
| | 2_1_153_CM1 | | | | | | VKCK |
| | VKCK IL- | IL-2Rα (1-238) | (GGGGS)2 (SEQ | CM1 | GGS | VHCH | IL-2Rα-L1- |
| | 2Rα_1_238_CM1_IL- | | ID NO: 12) | | | | CM1-L2-IL- |
| | 2Rβ_27_239_CM1 | IL-2Rβ (27-239) | GGGGS (SEQ ID | CM1 | GGS | | 2Rß-L3- |
| | VHCH | | NO: 10) | | | | CM1-L4- |
| | | | | | | | VHCH |
| 490 | pNOVI V2K | IL-2 (1-153) | GGGGS (SEQ ID | CM1 | GGS | VKCK | IL-2-L1- |
| | K91_C_IL- | | NO: 10) | | | | CM1-L2- |
| | 2_1_153_CM1 | | | | | | VKCK |
| | VKCK IL- | IL-2Rα (1-185) | GGGGS (SEQ ID | CM1 | GGS | VHCH | IL-2Rα-L1- |
| | 2Rα_1_185_CM1_IL- | | NO: 10) | | | | CM1-L2-IL- |
| | 2Rβ_27_239_CM1 | IL-2Rß (27-239) | GGGGS (SEQ ID | CM1 | GGS | | 2Rß-L3- |
| | VHCH | | NO: 10) | | | | CM1-L4- |
| | | | | | | | VHCH |
| 500 | pNOVI V2K | IL-2 (1-153) | GGGGS (SEQ ID | CM1 | GGGGS | VKCK | IL-2-L1- |
| | K91_D1_IL- | | NO: 10) | | (SEQ | | CM1-L2-IL- |
| | 2_1_153_CM1_IL- | | | | ID NO: | | 2Rß-L3- |
| | 2Rβ_27_239_CM1 | | | | 10) | | CM1-L4- |
| | VKCK IL- | IL-2Rß (27-239) | GGGGS (SEQ ID | CM1 | GGS | | VKCK |
| | 2Rα_1_238_CM1_ | | NO: 10) | | | | |
| | VHCH | IL-2Rα (1-238) | GGGGS (SEQ ID | CM1 | GGS | VHCH | IL-2Rα-L1- |
| | | | NO: 10) | | | | CM1-L2- |
| | | | | | | | VHCH |
| 651 | pNOVI V2K | IL-2Rα (1-238) | GGGGS (SEQ ID | CM1 | GGGGS | VKCK | IL-2Rα-L1- |
| | K91_E6_IL- | | NO: 10) | | (SEQ | | CM1-L2-IL- |
| | 2Rα_1_238_CM1_IL- | | | | ID NO: | | 2-L3-CM1- |
| | 2_21_153_CM1 | | | | 10) | | L4-VKCK |
| | VKCK IL- | IL-2 (21-153) | GGGGS (SEQ ID | CM1 | GGS | | |
| | 2Rβ_1_239_CM1_ | | NO: 10) | | | | |
| | VHCH | IL-2Rß (1-239) | GGGGS (SEQ ID | CM1 | GGS | VHCH | IL-2Rβ-L1- |
| | | | NO: 10) | | | | CM1-L2- |
| | | | | | | | VHCH |
| 676 | pNOVI V2K | IL-2Rα (1-238) | (GGGGS)2 | — | — | VKCK | IL-2Rα-L1- |
| | K91_E8_IL- | | (SEQ ID NO: 12) | | | | IL-2-L3- |
| | 2Rα_1_238_IL- | IL-2 (21-153) | GGGGS (SEQ ID | CM1 | GGS | | CM1-L4- |
| | 2_21_153_CM1 | | NO: 10) | | | | VKCK |
| | VKCK IL- | IL-2Rβ (1-239) | GGGGS (SEQ ID | CM1 | GGS | VHCH | IL-2Rβ-L1- |
| | 2Rβ_1_239_CM1_ | | NO: 10) | | | | CM1-L2- |
| | VHCH | | | | | | VHCH |
| 690 | pNOVI V2K K91_IL- | IL-2Rα (1-238) | GGGGS (SEQ ID | CM1 | GGS | VKCK | IL-2Rα-L1- |
| | 2Rα CM1_VKCK_IL- | | NO: 10) | | | | CM1-L2- |
| | 2_CM1_VHCH | | | | | | VKCK |
| | | IL-2 (1-153) | GGGGS (SEQ ID | CM1 | GGS | VHCH | IL-2-L1- |
| | | | NO: 10) | | | | CM1-L2- |
| | | | | | | | VHCH |
| 860 | pNOVI V2K K91_E6 | IL-2Rα (1-238) | GGGGSVHMGG | — | — | VKCK | IL-2Rα-L1- |
| | NO LIN_IL- | | S(GGGGS)2 | | | | IL-2-L3- |
| | 2Rα_1_238_CM1_IL- | | (SEQ ID NO: 13) | | | | VKCK |
| | 2_21_153_CM1 | IL-2 (21-153) | (GGGGS)3GGS | — | — | | |
| | VKCK IL- | | (SEQ ID NO: 14) | | | | |
| | 2Rβ_1_239_CM1_ | IL-2Rβ (1-239) | (GGGGS)3GGS | — | — | VHCH | IL-2Rβ-L1- |
| | VHCH | | (SEQ ID NO: 14) | | | | VHCH |
| 900 | pNOVI V2K K91_E8 | IL-2Rα (1-238) | GGGGSVHMGS | — | — | VKCK | IL-2Rα-L1- |
| | NO LIN_IL- | | (SEQ ID NO: 15) | | | | IL-2-L3- |
| | 2Rα_1_238_IL- | IL-2 (21-153) | (GGGGS)3GGS | — | — | | VKCK |
| | 2_21_153_CM1 | | (SEQ ID NO: 14) | | | | |
| | VKCK IL- | IL-2Rß (1-239) | (GGGGS)3GGS | — | — | VHCH | IL-2Rβ-L1- |
| | 2Rβ_1_239_CM1_ | | (SEQ ID NO: 14) | | | | VHCH |
| | VHCH | | | | | | |
| 830 | pNOVI V2K | IL-2Rα (1-238) | GGGGS (SEQ ID | CM1 | GGGGS | VKCK | IL-2Rα-L1- |
| | K33_E6_IL- | | NO: 10) | | (SEQ | | CM1-L2-IL- |
| | 2Rα_1_238_CM1_IL- | | | | ID NO: | | 2-L3-CM1- |
| | 2_21_153_CM1 | | | | 10) | | L4-VKCK |
| | VKCK IL- | IL-2 (21-153) | GGGGS (SEQ ID | CM1 | GGS | | |
| | 2Rβ_1_239_CM1_ | | NO: 10) | | | | |
| | VHCH | IL-2Rß (1-239) | GGGGS (SEQ ID | CM1 | GGS | VHCH | IL-2Rβ-L1- |
| | | | NO: 10) | | | | CM1-L2- |
| | | | | | | | VHCH |
| 770 | pNOVI V2K | IL-2Rα (1-238) | GGGGS (SEQ ID | CM1 | GGGGS | VKCK | IL-2Rα-L1- |
| | K91_M1_IL- | | NO: 10) | | (SEQ | | CM1-L2-IL- |
| | 2Rα_1_238_CM1_IL- | | | | ID NO: | | 2 C125S- |
| | 2 | | | | 10) | | L3-CM1- |
| | C125S_21_153_CM1 | IL-2 C125S | GGGGS (SEQ ID | CM1 | GGS | | L4-VKCK |
| | VKCK IL- | (21-153) | NO: 10) | | | | |
| | 2Rβ_1_239_CM1_ | IL-2Rß (1-239) | GGGGS (SEQ ID | CM1 | GGS | VHCH | IL-2Rβ-L1- |
| | VHCH | | NO: 10) | | | | CM1-L2- |
| | | | | | | | VHCH |
| 780 | pNOVI V2K | IL-2Rα (1-238) | GGGGS (SEQ ID | CM1 | GGGGS | VKCK | IL-2Rα-L1- |
| | K91_M2_IL- | | NO: 10) | | (SEQ | | CM1-L2-IL- |
| | 2Rα_1_238_CM1_IL- | | | | ID NO: | | 2 F42A-L3- |
| | 2 | | | | 10) | | CM1-L4- |
| | F42A 21_153_CM1 | IL-2 F42A | GGGGS (SEQ ID | CM1 | GGS | | VKCK |
| | VKCK IL- | (21-153) | NO: 10) | | | | |
| | 2Rβ_1_239_CM1_ | IL-2Rβ (1-239) | GGGGS (SEQ ID | CM1 | GGS | VHCH | IL-2Rβ-L1- |
| | VHCH | | NO: 10) | | | | CM1-L2- |
| | | | | | | | VHCH |
| 790 | pNOVI V2K | IL-2Rα (1-238) | GGGGS (SEQ ID | CM1 | GGGGS | VKCK | IL-2Rα-L1- |
| | K91_M3_IL- | | NO: 10) | | (SEQ | | CM1-L2-IL- |
| | 2Rα_1_238_CM1_IL- | | | | ID NO: | | 2 D20T-L3- |
| | 2 | | | | 10) | | CM1-L4- |
| | D20T_21_153_CM1 | IL-2 D20T | GGGGS (SEQ ID | CM1 | GGS | | VKCK |
| | VKCK IL- | (21-153) | NO: 10) | | | | |
| | 2Rβ_1_239_CM1_ | IL-2Rβ (1-239) | GGGGS (SEQ ID | CM1 | GGS | VHCH | IL-2Rβ-L1- |
| | VHCH | | NO: 10) | | | | CM1-L2- |
| | | | | | | | VHCH |
| 800 | pNOVI V2K | IL-2Rα (1-238) | GGGGS (SEQ ID | CM1 | GGGGS | VKCK | IL-2Rα-L1- |
| | K91_M4_IL- | | NO: 10) | | (SEQ | | CM1-L2-IL- |
| | 2Rα_1_238_CM1_IL- | | | | ID NO: | | 2 Q126T- |
| | 2 | | | | 10) | | L3-CM1- |
| | Q126T_21_153_CM1 | IL-2 Q126T | GGGGS (SEQ ID | CM1 | GGS | | L4-VKCK |
| | VKCK IL- | (21-153) | NO: 10) | | | | |
| | 2Rβ_1_239_CM1_ | IL-2Rß (1-239) | GGGGS (SEQ ID | CM1 | GGS | VHCH | IL-2Rβ-L1- |
| | VHCH | | NO: 10) | | | | CM1-L2- |
| | | | | | | | VHCH |
| 841 | pNOVI V2K L1_IL- | IL-2Rß (1-239) | GGGGS (SEQ ID | CM1 | GGS | VKCK | IL-2Rβ-L1- |
| | 2Rβ_1_239_CM1_IL | | NO: 10) | | | | CM1-L2-IL- |
| | 2Rα_22_238_CM1_ | IL-2Rα (22-238) | GGGGS (SEQ ID | CM1 | GGGGS | | 2Rα-L3- |
| | IL-2_21_153_CM1 | | NO: 10) | | (SEQ | | CM1-L4-IL- |
| | VKCK | | | | ID NO: | | 2-L5-CM1- |
| | | | | | 10) | | L6-VKCK |
| | | IL-2 (21-153) | GGGGS (SEQ ID | CM1 | GGS | | |
| | | | NO: 10) | | | | |
| | | — | — | — | — | VHCH | VHCH |
| 870 | pNOVI V2K | IL-2Rγ (1-262) | GGGGS (SEQ ID | CM1 | GGGGS | VKCK | IL-2Rγ-L1- |
| | K91_G3_IL- | | NO: 10) | | (SEQ | | CM1-L2-IL- |
| | 2Rγ_1_262_CM1_IL- | | | | ID NO: | | 2-L3-CM1- |
| | 2_21_153_CM1 | | | | 10) | | L4-VKCK |
| | VKCK IL- | IL-2 (21-153) | GGGGS (SEQ ID | CM1 | GGS | | |
| | 2Rα_1_238_CM1_ | | NO: 10) | | | | |
| | VHCH | IL-2Rα (1-238) | GGGGS (SEQ ID | CM1 | GGS | VHCH | IL-2Rα-L1- |
| | | | NO: 10) | | | | CM1-L2- |
| | | | | | | | VHCH |
| 880 | pNOVI V2K | IL-2Rγ (1-262) | (GGGGS)2 | — | — | VKCK | IL-2Rγ-L1- |
| | K91_G4_IL- | | (SEQ ID NO: 12) | | | | IL-2-L3- |
| | 2Rγ_1_262_IL- | IL-2 (21-153) | GGGGS (SEQ ID | CM1 | GGS | | CM1-L4- |
| | 2_21_153_CM1 | | NO: 10) | | | | VKCK |
| | VKCK_IL- | IL-2Rα (1-238) | GGGGS (SEQ ID | CM1 | GGS | VHCH | IL-2Rα-L1- |
| | 2Rα_1_238_CM1_ | | NO: 10) | | | | CM1-L2- |
| | VHCH | | | | | | VHCH |
| 1021 | pNOVI V2K | IL-2Rα (1-238) | GGGGS (SEQ ID | CM1 | GGGGS | VKCK | IL-2Rα-L1- |
| | K91_J1_IL- | | NO: 10) | | (SEQ | | CM1-L2-IL- |
| | 2Rα_1_238_CM1_IL- | | | | ID NO: | | 2-L3-CM1- |
| | 2_21_153_CM1 | | | | 10) | | L4-VKCK |
| | VKCK_IL- | IL-2 (21-153) | GGGGS (SEQ ID | CM1 | GGS | | |
| | 2Rα_1_238_CM1_IL- | | NO: 10) | | | | |
| | 2_21_153_CM1 | IL-2Rα (1-238) | GGGGS (SEQ ID | CM1 | GGGGS | VHCH | IL-2Rα-L1- |
| | VHCH | | NO: 10) | | (SEQ | | CM1-L2-IL- |
| | | | | | ID NO: | | 2-L3-CM1- |
| | | | | | 10) | | L4-VHCH |
| | | IL-2 (21-153) | GGGGS (SEQ ID | CM1 | GGS | | |
| | | | NO: 10) | | | | |
| 1051 | pNOVI V2K | IL-2Rα (1-238) | (GGGGS)2 | — | — | VKCK | IL-2Rα-L1- |
| | K91_J3_IL- | | (SEQ ID NO: 12) | | | | IL-2-L3- |
| | 2Rα_1_238_IL- | IL-2 (21-153) | GGGGS (SEQ ID | CM1 | GGS | | CM1-L4- |
| | 2_21_153_CM1 | | NO: 10) | | | | VKCK |
| | VKCK_IL- | IL-2Rα (1-238) | (GGGGS)2 | — | — | VHCH | IL-2Rα-L1- |
| | 2Rα_1_238_IL- | | (SEQ ID NO: 12) | | | | L2-IL-2-L3- |
| | 2_21_153_CM1 | IL-2 (21-153) | GGGGS (SEQ ID | CM1 | GGS | | CM1-L4- |
| | VHCH | | NO: 10) | | | | VHCH |
| 1030 | pNOVI V2K | IL-2 Q126T | GGGGS (SEQ ID | CM1 | GGS | VKCK | IL-2 |
| | K91_Q1_IL-2 | (1-153) | NO: 10) | | | | Q126T-L1- |
| | Q126T | | | | | | CM1-L2- |
| | CM1_VKCK_IL- | | | | | | VKCK |
| | 2Rα_CM1_VHCH | IL-2Rα (1-238) | GGGGS (SEQ ID | CM1 | GGS | VHCH | IL-2Rα-L1- |
| | | | NO: 10) | | | | CM1-L2- |
| | | | | | | | VHCH |

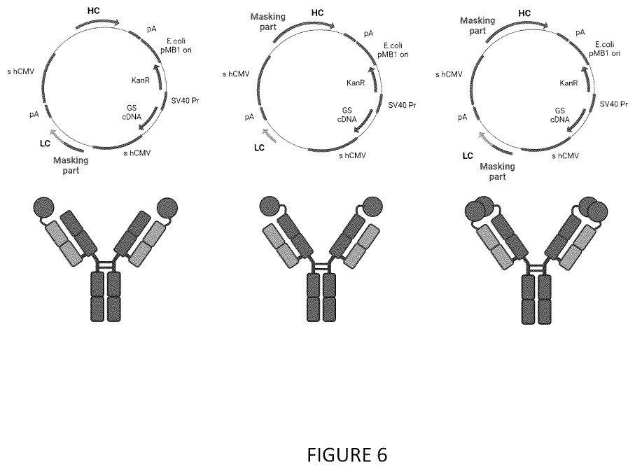

To generate the antibody-cytokine/receptor fusions, expression plasmids encoding these different constructs were generated. The expression vector contains an origin of replication, a kanamycin resistance gene, and two expression cassettes under the transcription control of human cytomegalovirus promoter (hCMV) for expression in mammalian cells of HC and LC with or without a cleavable masking domain. A SV40 promoter and glutamine synthetase gene were also present for expression in CHO cells. Examples of expression vectors and illustrations of the corresponding configurations are shown in FIG. 6. Synthetic sequences, composed of a masking domain, a flexible linker, a cleavable linker, and another flexible linker, were purchased from Eurofins and flanked by appropriate restriction enzyme sites for molecular cloning into the expression vectors. 10 μg of the expression vector pNOVI K91 (allowing for the expression of the high affinity anti-CD47 antibody K91) or pNOVI K33 (allowing for the expression of the low affinity anti-CD47 antibody K33) and 10 μg of the synthetic DNA insert encoding the different constructs listed in Table 1 were digested with 10 units of the appropriate restriction enzymes for 1 h at 37° C. The digested vectors were dephosphorylated by incubation with 40 units of phosphatase alkaline for 15 min at 37° C. Vectors and inserts were then loaded on E-Gel Agarose with SYBR Safe DNA Gel Stain, 1.2% (Invitrogen) and purified twice using MiniElute Gel Extraction Kit (Qiagen) according to Qiagen protocol. After purification 15 to 30 ng of insert DNA were ligated into 40 to 50 ng of vector using Rapid DNA Ligation Kit (Roche). Ligation controls were performed with DNA vector alone and DNA insert alone. 30 μL of E. coli XLI competent cells were thawed slowly on ice and added to the ligation reaction and kept on ice for 30 min. Then, a heat shock was performed by incubating cells for 1 min at 42° C. and then for 2 min on ice. Cells allowed to recover in 500 μL of SOC medium (Invitrogen) for 1 h at 37° C. with agitation at 1250 rpm. Cells were then spread onto LB agar plate containing kanamycin and incubated at 37° C., ON. Some colonies were randomly picked, plated on a Masterplate to isolate each clone and cultured in 5 mL of LB medium with kanamycin at 37° C., ON. DNA was extracted using QIAprep Spin Miniprep Kit (Qiagen). Analytical DNA digestion was performed to check the presence of the insert, followed by sequencing of clones with expected insert size. A mix with around 200 ng of DNA and 1 μM of primers in a 5 μL final volume was used for Sanger sequencing. Sequences were aligned with the theoretical reference sequences using Sequencher software. Clones having a correct sequence were inoculated at 37° C., ON. DNA was extracted and purified using PureLink HiPure Plasmid Filter DNA Purification Kit (Invitrogen), according to Invitrogen Maxiprep protocol. Purified DNA was sequenced as mentioned above.

### Example 2. Expression and Purification of Antibody-Cytokine/Receptor Fusions

The expression vectors of Example 1 were transiently transfected into Expi293 cells, and the corresponding antibody-cytokine/receptor fusions were purified and characterized. Expi293 were cultured in Expi293 Expression Medium (ThermoFisher) containing 25 mg/L gentamicin (Gibco) at 37° C., with >80% relative humidity, 8% CO2 under agitation at 120 rpm. On the day of transfection, cells were diluted at 3×10⁶ cells/mL. 50 mL of cells were transfected using polyethylenimine (PEI) (Polysciences) transfection reagent. A DNA mix was prepared with 1.3 mL of NaCl and 62.5 μg of DNA. The DNA mix was added drop by drop to a PEI mix prepared with 1.3 mL of NaCl and 250 μL of PEI and incubated for 10 min at RT. Then, the mix DNA/PEI was transferred drop by drop to the Expi293 cells. The transfected cells were incubated at 37° C., with >80% relative humidity, 8% CO2 under agitation at 120 rpm. After 6 days of culture, supernatant was recovered and filtered on 0.22 μm membrane using Sartoclear Dynamics Lab V kit (Sartorius). Antibodies were purified by affinity chromatography using FcXL affinity matrix (ThermoFisher). An appropriate amount of FcXL resin pre-washed 3 times in phosphate-buffered saline (PBS) was added to the supernatant and incubated at 4ºC, 15 rpm, ON. Then samples were centrifuged for 10 min at 2000 rpm and 4° C. to recover the resin and the flow through was discarded. Resin was washed twice with PBS, transferred to an Amicon Pro device (Merck), washed again with PBS and centrifuged 5 min at 200 g. Then, elution was performed with 50 mM glycine pH3.5 elution buffer neutralized with 1/10 (v:v) of 1M Tris-HCl pH7.5. Three elution fractions of 3 mL were applied, recovered, and transferred on a pre-equilibrated Amicon membrane 50 kDa (Merck) with 25 mM Histidine, 125 mM NaCl pH6.0 formulation buffer. 3 steps of dilution/concentration with formulation buffer were performed with centrifugation at 3500 rpm between each step. Then the desalted and concentrated sample was recovered and transferred in a LoBind tube (Eppendorf). Antibody concentration was measured by Nanodrop.

### Example 3. Characterization of Antibody-Cytokine/Receptor Fusions

The purity, molecular size of the antibody-cytokine/receptor fusions and integrity were evaluated by SDS-PAGE. Purified antibody-cytokine/receptor fusions were loaded on NuPAGE gel (Invitrogen) under denaturing and reducing conditions. 5 μg of protein diluted in PBS were incubated with NuPAGE LDS 4× buffer (Invitrogen) containing 4% β-mercaptoethanol for 5 min at 95° C. The migration was performed in 1×MES NuPAGE running buffer (Invitrogen) for 45 min at 150V. Gel was stained with Coomassie blue for antibody integrity and aggregation state were assessed by SEC-UPLC with an Acquity UPLC BEH SEC column (Waters) using a 0.2M sodium phosphate pH 6.8 mobile phase.

The characterization of the different antibody-cytokine/receptor fusions is summarized in Table 2.

TABLE 2
Yields of antibody-cytokine/receptor fusions and aggregate levels.

| | | Characterization | |
|---|---|---|---|
| Construct | | Quantity | Aggregates |
| No | Name | (μg) | (%) |
| 46 | pNOVI V2K K91 | 5430 | 3.25 |
| 22 | pNOVI V2K K33 | 4954 | 3.79 |
| 2 | pNOVI V2K K91_IL-2 CM1_VKCK | 1266 | 3.77 |
| 5 | pNOVI V2K K91_IL-2 CM1_CM2_VKCK | 3976 | 1.64 |
| 13 | pNOVI V2K K91_IL-2 CM1_VHCH | 1382 | 6.04 |
| 17 | pNOVI V2K K91_IL-2 CM1_CM2_VHCH | 2256 | 2.53 |
| 31 | pNOVI V2K K91_IL-2 CM1_VKCK_IL-2_CM1_VHCH | 121 | 53.02 |
| 41 | pNOVI V2K K91_IL-2 CM1_VKCK_IL-2Rα_CM1_VHCH | 2324 | 0.79 |
| 71 | pNOVI V2K K91_IL-2_CM1_CM2_VKCK_IL- | 69 | 42.27 |
| | 2_CM1_CM2_VHCH | | |
| 9 | pNOVI V2K K91_IL15_CM1_VKCK | 26 | ND |
| 111 | pNOVI V2K K91_IL15_CM1_VHCH | 279 | 48.57 |
| 52 | pNOVI V2K K91_IL15_CM1_VKCK_IL15_CM1_VHCH | 16 | 59.07 |
| 81 | pNOVI V2K K91_IL15_CM1_VKCK_IL15RA_Sushi | 623 | 34.8 |
| | 1_CM1_VHCH | | |
| 281 | pNOVI V2K K91_IL-2_CM1_VKCK_IL-2Rα WO LIN_VHCH | 3212 | 1.24 |
| 291 | pNOVI V2K K91_IL-2_CM1_VKCK_IL-2Rα WO LIN | 1995 | 1.36 |
| | CM1_VHCH | | |
| 361 | pNOVI V2K K91_IL-2 WO LIN CM1 VKCK_IL-2Rα WO LIN | 2908 | 1.51 |
| | CM1_VHCH | | |
| 450 | pNOVI V2K K91_A_IL-2_1_153_CM1 VKCK_IL- | 651 | ND |
| | 2Rα_1_238_CM1_IL-2Rβ_27_239_CM1 VHCH | | |
| 471 | pNOVI V2K K91_A1_IL-2_1_153_CM1 VKCK IL- | 558 | ND |
| | 2Rα_1_238_CM1_IL-2Rβ_27_239_CM1 VHCH | | |
| 530 | pNOVI V2K K91_A2_IL-2_1_153_CM1 VKCK IL- | 418 | ND |
| | 2Rα_1_238_CM1_IL-2Rβ_27_239_CM1 VHCH | | |
| 545 | pNOVI V2K K91_A3_IL-2_1_153_CM1 VKCK IL- | 504 | ND |
| | 2Rα_1_238_CM1_IL-2Rβ_27_239_CM1 VHCH | | |
| 490 | pNOVI V2K K91_C_IL-2_1_153_CM1 VKCK IL- | 448 | ND |
| | 2Rα_1_185_CM1_IL-2Rβ_27_239_CM1 VHCH | | |
| 500 | pNOVI V2K K91_D1_IL-2_1_153_CM1_IL-2Rβ_27_239_CM1 | 678 | ND |
| | VKCK IL-2Rα_1_238_CM1_VHCH | | |
| 651 | pNOVI V2K K91_E6_IL-2Rα_1_238_CM1_IL-2_21_153_CM1 | 498 | ND |
| | VKCK IL-2Rβ_1_239_CM1_VHCH | | |
| 676 | pNOVI V2K K91_E8_IL-2Rα_1_238_IL-2_21_153_CM1 VKCK | 727 | ND |
| | IL-2Rβ_1_239_CM1_VHCH | | |
| 690 | pNOVI V2K K91_IL-2Rα CM1_VKCK_IL-2_CM1_VHCH | 1040 | ND |
| 860 | pNOVI V2K K91_E6 NO LIN_IL-2Rα_1_238_CM1_IL- | 733 | ND |
| | 2_21_153_CM1 VKCK IL-2Rβ_1_239_CM1_VHCH | | |
| 900 | pNOVI V2K K91_E8 NO LIN_IL-2Rα_1_238_IL-2_21_153_CM1 | 1086 | ND |
| | VKCK IL-2Rβ_1_239_CM1_VHCH | | |
| 830 | pNOVI V2K K33_E6_IL-2Rα_1_238_CM1_IL-2_21_153_CM1 | 219 | ND |
| | VKCK IL-2Rβ_1_239_CM1_VHCH | | |
| 770 | pNOVI V2K K91_M1_IL-2Rα_1_238_CM1_IL-2 | 478 | ND |
| | C125S_21_153_CM1 VKCK IL-2Rβ_1_239_CM1_VHCH | | |
| 780 | pNOVI V2K K91_M2_IL-2Rα_1_238_CM1_IL-2 | 605 | ND |
| | F42A_21_153_CM1 VKCK IL-2Rβ_1_239_CM1_VHCH | | |
| 790 | pNOVI V2K K91_M3_IL-2Rα_1_238_CM1_IL-2 | 181 | ND |
| | D20T_21_153_CM1 VKCK IL-2Rβ_1_239_CM1_VHCH | | |
| 800 | pNOVI V2K K91_M4_IL-2Rα_1_238_CM1_IL-2 | 594 | ND |
| | Q126T_21_153_CM1 VKCK IL-2Rβ_1_239_CM1_VHCH | | |
| 841 | pNOVI V2K L1_IL-2Rβ_1_239_CM1_IL-2Rα_22_238_CM1_IL- | 260 | ND |
| | 2_21_153_CM1 VKCK | | |
| 870 | pNOVI V2K K91_G3_IL-2Rγ_1_262_CM1_IL-2_21_153_CM1 | 159 | ND |
| | VKCK_IL-2Rα_1_238_CM1_VHCH | | |
| 880 | pNOVI V2K K91_G4_IL-2Rγ_1_262_IL-2_21_153_CM1 | 367 | ND |
| | VKCK_IL-2Rα_1_238_CM1_VHCH | | |
| 1021 | pNOVI V2K K91_J1_IL-2Rα_1_238_CM1_IL-2_21_153_CM1 | 63 | ND |
| | VKCK_IL-2Rα_1_238_CM1_IL-2_21_153_CM1 VHCH | | |
| 1051 | pNOVI V2K K91_J3_IL-2Rα_1_238_IL-2_21_153_CM1 | 324 | ND |
| | VKCK_IL-2Rα_1_238_IL-2_21_153_CM1 VHCH | | |
| 1030 | pNOVI V2K K91_Q1_IL-2 Q126T CM1_VKCK_IL- | 2419 | ND |
| | 2Rα_CM1_VHCH | | |

Overall constructs with IL-15 alone on the LC or on the LC and the HC showed lower expression level. A better expression level was observed with combined masking of IL-15 on the LC and IL-15Rα on the HC suggesting a better stability thanks to the possible interaction between IL-15 and IL-15Rα. Masked constructs with IL-2 or IL-2Rα showed good expression level. Constructs with IL-2 fused to both LC and HC showed a high aggregate level (42%, 53%). These constructs also displayed unexpected patterns on SDS-PAGE analysis. Constructs with IL-2 on the LC and IL-2Rα on the HC showed good expression and low aggregate level (<1%) suggesting a good stability thanks to the possible interaction between IL-2 and IL-2Rα. Similar observations could be made for other constructs containing several extracellular domains of the cytokine receptor that could be expressed and purified (Table 2). For a number of constructs, as their molecular weight is significantly different from an IgG, the aggregates levels could not be determined as they showed an unusual profile on SEC-UPLC (indicated as ND in Table 2).

Based on this initial characterization antibody-cytokine/receptor fusions were selected for further biological characterization.

### Example 4. Proteolytic Cleavage of the Masking Domains

Proteolytic cleavage of antibody-cytokine/receptor fusions was evaluated. 3 μg of antibodies were treated with 10 units of hMMP-9 (Abcam) in the reaction buffer containing 50 mM Tris, 150 mM NaCl, 5 mM CaCl2, 20 μM ZnCl2, pH7.5 in a final volume of 20 μL. Reactions were carried out at 37ºC for 4 h to 5 h. Cleavage of masking domains was evaluated by SDS-PAGE analysis in reducing and denaturing conditions as previously described.

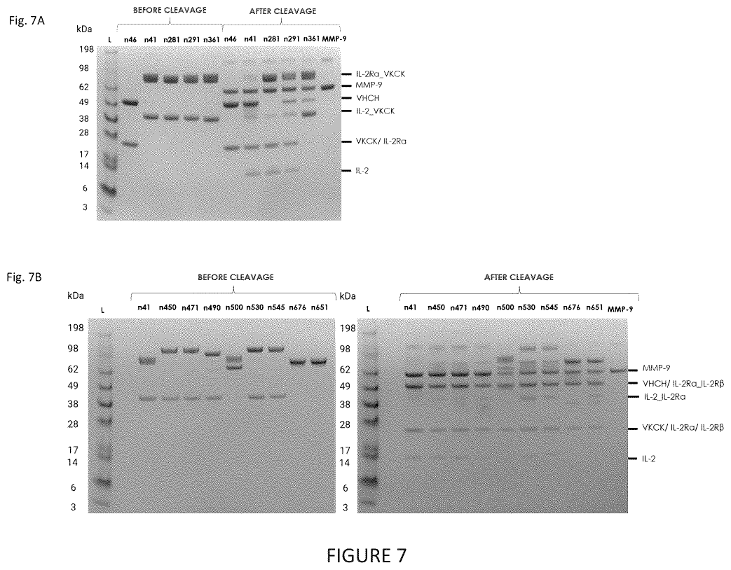

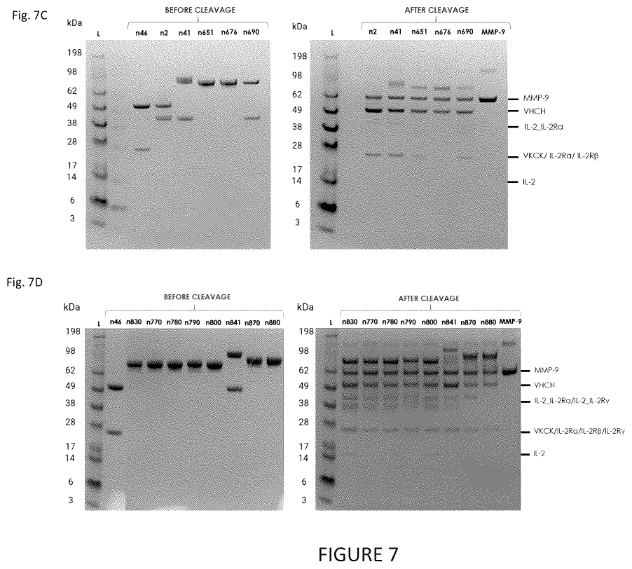

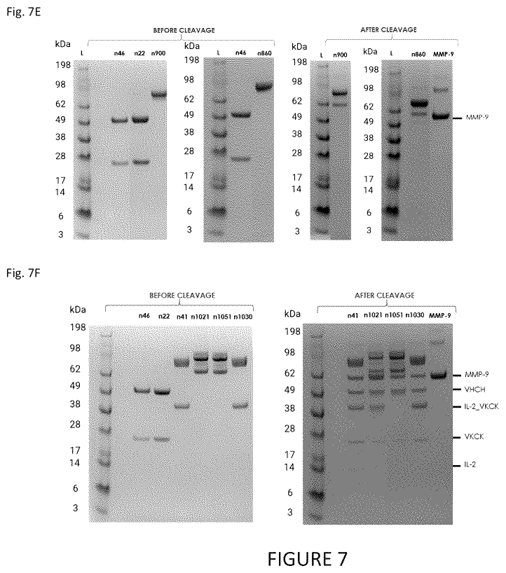

Antibody-cytokine/receptor fusions were incubated with recombinant MMP-9 and cleavage efficacy was visualized by SDS-PAGE in denaturing and reducing conditions (FIG. 7). The mAbs K91 (n46) and K33 (n22) were used as control. As shown, before cleavage mAbs K91 and K33 showed two bands corresponding to HC (˜48 kDa) and LC (˜23 kDa) (FIGS. 7A and 7C-7E and FIG. 7E respectively). After MMP-9 treatment (˜60 kDa), n361 was not effectively cleaved with still the presence of bands corresponding to masked HC and LC with strong intensity. In contrast n41, n281 and n291 were more effectively cleaved with the appearance of bands corresponding to unmasked HC and LC, as well as one band that probably corresponds to IL-2 (˜15 kDa). IL-2Rα, not visible on the gel may have co-migrated with the LC, their molecular weight being similar (˜24 kDa for IL-2Rα and ˜23 kDa for the LC) (FIG. 7A). Overall, all constructs could be cleaved by the protease with expected bands appearing of SDS-PAGE gels (FIGS. 7B-7D and 7F). As expected, non cleavable constructs n860 and n900 remained intact after protease cleavage (FIG. 7E).

### Example 5. Antibody Binding Profiles Before and After Proteolytic Cleavage of Antibody-Cytokine/Receptor Fusions

The capacity of the masking domains to inhibit binding activity of the antibody was evaluated by Bio-Layer Interferometry (BLI). As positive control, wild type mAbs K91 (n46) and K33 (n22) were used. Binding experiments were performed to evaluate the masking efficiency and the binding recovery after cleavage of the masking domains. BLI was performed on an Octet RED96 system (Sartorius). His-tagged human CD47 was diluted at 2.5 μg/mL in Kinetic Buffer (KB) (Sartorius) and loaded on HIS1K biosensors (anti-His tag antibody biosensors, Sartorius) for 300 seconds. Loaded biosensors were dipped into antibodies diluted at 15 μg/mL in KB for 300 seconds to monitor association. Then, biosensors were transferred in KB for dissociation for 60 seconds. Binding profiles were then analyzed with ForteBio Data Analysis software.

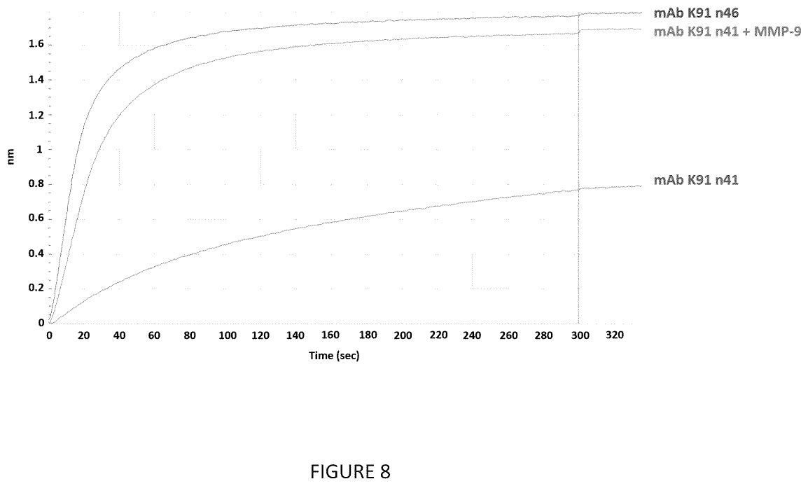

An example of binding profiles obtained with cleaved and non-cleaved antibody-cytokine/receptor fusions are shown in FIG. 8. The binding capacity for all constructs is shown in Table 3. Some constructs were not characterized after cleavage (indicated as ND).

All constructs showed lower association to the target antigen than the control antibody. Moreover, masking efficacy was generally correlated with molecular weights of masking domains, consistent with steric hindrance of the masking components.

Binding activity of antibody-cytokine/receptor fusions before and after cleavage was also evaluated by flow cytometry. Peak cells derived from HEK 293 which are human cells expressing CD47 were used to evaluate masking efficacy and binding recovery to CD47 by flow cytometry. Peak cells were diluted in cold FACS buffer (PBS, 2% BSA) to 1.2×10⁶ cells/mL. 3×10⁵ cells/well were added in a 96 well V-bottom plate. The plate was then centrifugated for 5 min at 1300 rpm, 4° C. Supernatants were removed and cells were washed twice using FACS buffer. A serial dilution of antibody-cytokine/receptor fusions previously digested with MMP-9 or not digested as described above was prepared in FACS buffer. An irrelevant antibody was included. Then, 150 μL of each diluted antibody were added in corresponding well and incubated for 30 min at 4° C. Cells were washed twice with FACS buffer and 100 μL of a mouse anti human Fc-PE conjugated secondary antibody were added and the plate was incubated for 20 min at 4° C. Cells were then washed twice with FACS buffer. Finally, 150 μL of SYTOX blue dead cell stain (ThermoFisher) diluted to 1/5000 in FACS buffer were added before detection by flow cytometry with Cytoflex (Beckman Coulter). Acquisition was performed with 10000 events for each well. Data were analyzed by FlowJo software.

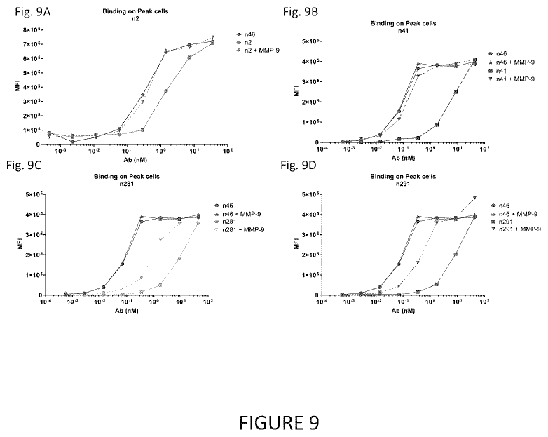

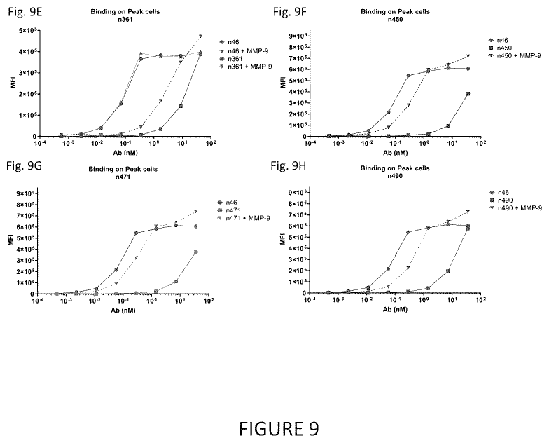

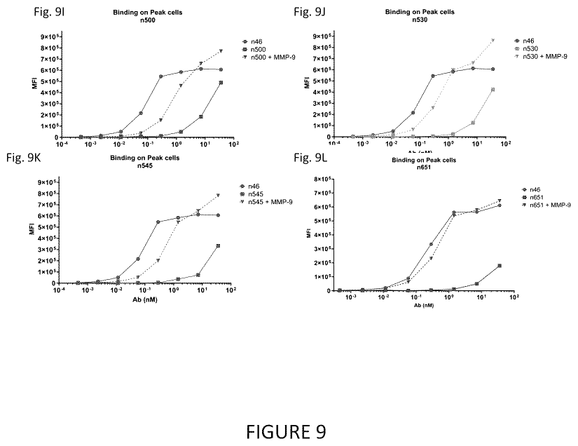

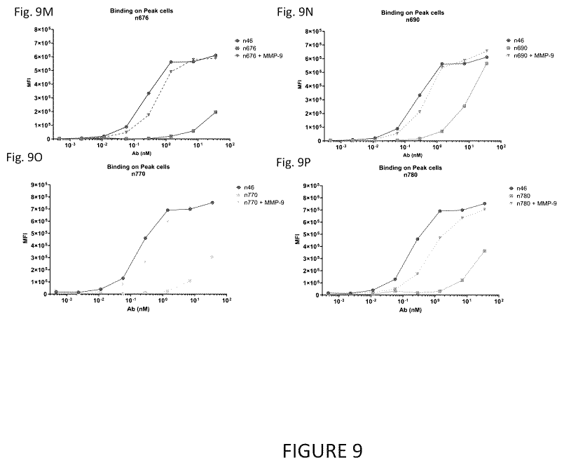

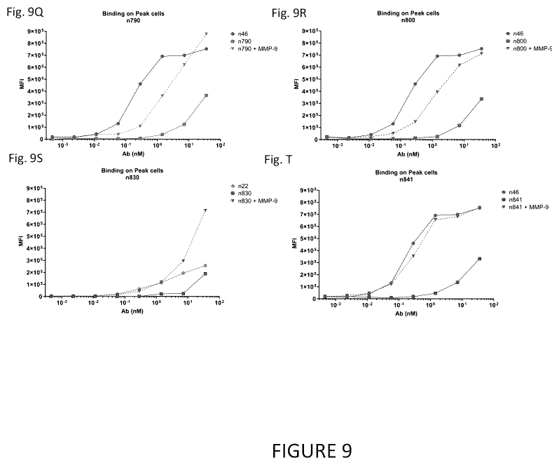

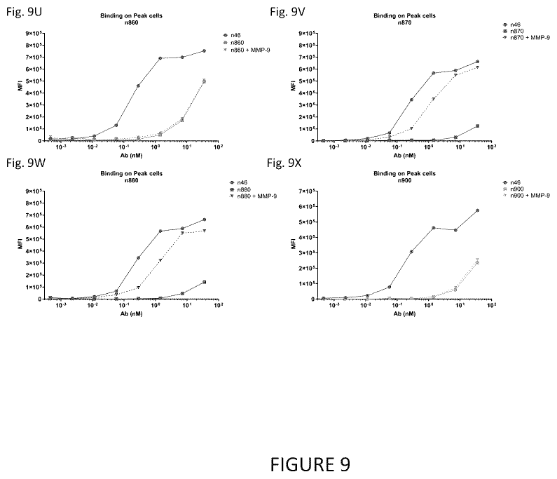

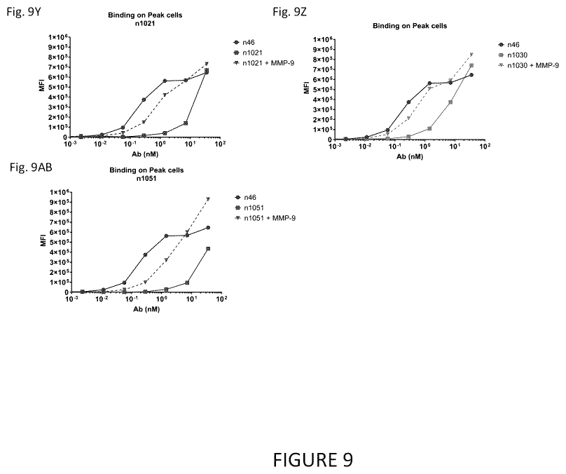

The binding to cell surface CD47 of the antibody-cytokine/receptor fusions corresponding to different constructs before and after proteolytic cleavage is shown in FIG. 9.

As expected, wild type antibody K91 n46 showed strong binding to Peak cells before and after proteolytic cleavage (FIGS. 9A-9R and 9T-9AB). In contrast the lower affinity anti-CD47 wild type antibody K33 n22 showed much lower binding levels (FIG. 9S). All antibody-cytokine/receptor fusions showed strong reduction of binding on Peak cells before cleavage with a decrease in the EC50 for binding compared to mAb K91 n46 (FIG. 9B-9AB). Masked n2 showed weaker binding reduction (FIG. 9A). After cleavage, the unmasked antibody showed different levels of binding recovery. Moreover, constructs that did not contain cleavable linker (n860 and n900), showed a reduced binding profile that was not changed by proteolytic cleavage (FIGS. 9U and 9X).

Binding on Peak cell surface confirmed the masking efficacy provided by masking domains fused to the N-terminal mAb via steric hindrance. The GS linkers are required to enable efficient cleavage and an effective binding recovery.

TABLE 3
Binding profile of masked cytokine-antibody constructs. Masking of antibody binding was evaluated
by Bio-Layer Interferometry (BLI) technology and by flow cytometry. By flow cytometry, binding recovery
was evaluated by dividing the EC₅₀ before cleavage and after cleavage.

| | | Binding profile | | | | |
|---|---|---|---|---|---|---|
| | | Bio-Layer | | | | |
| | | Interferometry | | Flow cytometry | | |
| | | (Octet RED96) | | (CytoFLEX) | | |
| | | Binding | Binding | EC50 | EC50 | |
| | | before | after | before | after | Binding |
| Construct | | cleavage | cleavage | cleavage | cleavage | recovery |
| No | Name | (nm) | (nm) | (nM) | (nM) | (BC/AC) |
| 46 | pNOVI V2K K91 | 1.8 | — | 0.062 | — | — |
| 22 | pNOVI V2K K33 | 1.3 | — | ND | ND | — |
| 2 | pNOVI V2K K91_IL-2 CM1_VKCK | 2.3 | ND | 1.6 | 0.4 | 4 |
| 5 | pNOVI V2K K91_IL-2 | 2.2 | ND | ND | ND | |
| | CM1_CM2_VKCK | | | | | |
| 13 | pNOVI V2K K91_IL-2 CM1_VHCH | 2.3 | ND | ND | ND | — |
| 17 | pNOVI V2K K91_IL-2 | 2.2 | ND | ND | ND | — |
| | CM1_CM2_VHCH | | | | | |
| 31 | pNOVI V2K K91_IL-2 | 1.0 | ND | ND | ND | — |
| | CM1_VKCK_IL-2_CM1_VHCH | | | | | |
| 41 | pNOVI V2K K91_IL-2 | 0.9 | 1.7 | 5.5 | 0.2 | 31 |
| | CM1_VKCK_IL-2Rα_CM1_VHCH | | | | | |
| 71 | pNOVI V2K K91_IL- | 1.0 | ND | ND | ND | — |
| | 2_CM1_CM2_VKCK_IL- | | | | | |
| | 2_CM1_CM2_VHCH | | | | | |
| 9 | pNOVI V2K K91_IL15_CM1_VKCK | ND | ND | ND | ND | — |
| 111 | pNOVI V2K K91_IL15_CM1_VHCH | 1.5 | ND | ND | ND | — |
| 52 | pNOVI V2K | 0.1 | ND | ND | ND | — |
| | K91_IL15_CM1_VKCK_IL15_CM1_{—} | | | | | |
| | VHCH | | | | | |
| 81 | pNOVI V2K | 0.9 | ND | ND | ND | — |
| | K91_IL15_CM1_VKCK_IL15RA_{—} | | | | | |
| | Sushi 1_CM1_VHCH | | | | | |
| 281 | pNOVI V2K K91_IL- | 1.0 | ND | 8.5 | 0.9 | 9 |
| | 2_CM1_VKCK_IL-2Rα WO | | | | | |
| | LIN_VHCH | | | | | |
| 291 | pNOVI V2K K91_IL- | 0.8 | ND | 6.9 | 0.4 | 18 |
| | 2_CM1_VKCK_IL-2Rα WO LIN | | | | | |
| | CM1_VHCH | | | | | |
| 361 | pNOVI V2K K91_IL-2 WO LIN CM1 | 0.6 | 0.5 | 9.9 | 1.9 | 5 |
| | VKCK_IL-2Rα WO LIN CM1_VHCH | | | | | |
| 450 | pNOVI V2K K91_A_IL- | 0.6 | ND | 31.6 | 0.4 | 80 |
| | 2_1_153_CM1 VKCK_IL- | | | | | |
| | 2Rα_1_238_CM1_IL- | | | | | |
| | 2Rβ_27_239_CM1 VHCH | | | | | |
| 471 | pNOVI V2K K91_A1_IL- | 0.6 | ND | 31.8 | 0.3 | 96 |
| | 2_1_153_CM1 VKCK IL- | | | | | |
| | 2Rα_1_238_CM1_IL- | | | | | |
| | 2Rβ_27_239_CM1 VHCH | | | | | |
| 530 | pNOVI V2K K91_A2_IL- | 0.7 | ND | 25.7 | 0.4 | 61 |
| | 2_1_153_CM1 VKCK IL- | | | | | |
| | 2Rα_1_238_CM1_IL- | | | | | |
| | 2Rβ_27_239_CM1 VHCH | | | | | |
| 545 | pNOVI V2K K91_A3_IL- | 0.6 | ND | 39.4 | 0.6 | 68 |
| | 2_1_153_CM1 VKCK IL- | | | | | |
| | 2Rα_1_238_CM1_IL- | | | | | |
| | 2Rβ_27_239_CM1 VHCH | | | | | |
| 490 | pNOVI V2K K91_C_IL- | 0.7 | ND | 13.4 | 0.5 | 27 |
| | 2_1_153_CM1 VKCK IL- | | | | | |
| | 2Rα_1_185_CM1_IL- | | | | | |
| | 2Rβ_27_239_CM1 VHCH | | | | | |
| 500 | pNOVI V2K K91_D1_IL- | 0.7 | ND | 17.5 | 0.8 | 21 |
| | 2_1_153_CM1_IL- | | | | | |
| | 2Rβ_27_239_CM1 VKCK IL- | | | | | |
| | 2Rα_1_238_CM1_VHCH | | | | | |
| 651 | pNOVI V2K K91_E6_IL- | 0.4 | 1.6 | 68.3 | 0.7 | 95 |
| | 2Rα_1_238_CM1_IL- | | | | | |
| | 2_21_153_CM1 VKCK IL- | | | | | |
| | 2Rβ_1_239_CM1_VHCH | | | | | |
| 676 | pNOVI V2K K91_E8_IL- | 0.6 | 1.5 | 56.7 | 1.0 | 57 |
| | 2Rα_1_238_IL-2_21_153_CM1 | | | | | |
| | VKCK IL-2Rβ_1_239_CM1_VHCH | | | | | |
| 690 | pNOVI V2K K91_IL-2Rα | 1.2 | ND | 8.7 | 0.4 | 20 |
| | CM1_VKCK_IL-2_CM1_VHCH | | | | | |
| 860 | pNOVI V2K K91_E6 NO LIN_IL- | 0.5 | 0.6 | 22.5 | 20.7 | 1 |
| | 2Rα_1_238_CM1_IL- | | | | | |
| | 2_21_153_CM1 VKCK IL- | | | | | |
| | 2Rβ_1_239_CM1_VHCH | | | | | |
| 900 | pNOVI V2K K91_E8 NO LIN_IL- | 0.5 | 0.5 | 55.3 | 47.0 | 1 |
| | 2Rα_1_238_IL-2_21_153_CM1 | | | | | |
| | VKCK IL-2Rβ_1_239_CM1_VHCH | | | | | |
| 830 | pNOVI V2K K33_E6_IL- | 0.6 | 1.7 | ND | ND | — |
| | 2Rα_1_238_CM1_IL- | | | | | |
| | 2_21_153_CM1 VKCK IL- | | | | | |
| | 2Rβ_1_239_CM1_VHCH | | | | | |
| 770 | pNOVI V2K K91_M1_IL- | 0.4 | 1.7 | 54.4 | 0.5 | 106 |
| | 2Rα_1_238_CM1_IL-2 | | | | | |
| | C125S_21_153_CM1 VKCK IL- | | | | | |
| | 2Rβ_1_239_CM1_VHCH | | | | | |
| 780 | pNOVI V2K K91_M2_IL- | 0.5 | 1.7 | 41.2 | 1.0 | 40 |
| | 2Rα_1_238_CM1_IL-2 | | | | | |
| | F42A_21_153_CM1 VKCK IL- | | | | | |
| | 2Rβ_1_239_CM1_VHCH | | | | | |
| 790 | pNOVI V2K K91_M3_IL- | 0.6 | 1.4 | 40.5 | 1.6 | 25 |
| | 2Rα_1_238_CM1_IL-2 | | | | | |
| | D20T_21_153_CM1 VKCK IL- | | | | | |
| | 2Rβ_1_239_CM1_VHCH | | | | | |
| 800 | pNOVI V2K K91_M4_IL- | 0.4 | 1.5 | 47.0 | 1.5 | 32 |
| | 2Rα_1_238_CM1_IL-2 | | | | | |
| | Q126T_21_153_CM1 VKCK IL- | | | | | |
| | 2Rβ_1_239_CM1_VHCH | | | | | |
| 841 | pNOVI V2K L1_IL- | 0.7 | 1.7 | 45.4 | 0.3 | 138 |
| | 2Rβ_1_239_CM1_IL- | | | | | |
| | 2Rα_22_238_CM1_IL- | | | | | |
| | 2_21_153_CM1 VKCK | | | | | |
| 870 | pNOVI V2K K91_G3_IL- | 0.3 | 1.3 | 219.2 | 1.3 | 170 |
| | 2Rγ_1_262_CM1_IL- | | | | | |
| | 2_21_153_CM1 VKCK_IL- | | | | | |
| | 2Rα_1_238_CM1_VHCH | | | | | |
| 880 | pNOVI V2K K91_G4_IL- | 0.2 | 1.1 | 458.6 | 2.6 | 178 |
| | 2Rγ_1_262_IL-2_21_153_CM1 | | | | | |
| | VKCK_IL-2Rα_1_238_CM1_VHCH | | | | | |
| 1021 | pNOVI V2K K91_J1_IL- | 0.2 | 1.3 | 14.3 | 1.3 | 38 |
| | 2Rα_1_238_CM1_IL- | | | | | |
| | 2_21_153_CM1 VKCK_IL- | | | | | |
| | 2Rα_1_238_CM1_IL- | | | | | |
| | 2_21_153_CM1 VHCH | | | | | |
| 1051 | pNOVI V2K K91_J3_IL- | 0.1 | 1.2 | 29.9 | 1.7 | 80 |
| | 2Rα_1_238_IL-2_21_153_CM1 | | | | | |
| | VKCK_IL-2Rα_1_238_IL- | | | | | |
| | 2_21_153_CM1 VHCH | | | | | |
| 1030 | pNOVI V2K K91_Q1_IL-2 Q126T | 0.7 | 1.3 | 6.3 | 0.8 | 17 |
| | CM1_VKCK_IL-2Rα_CM1_VHCH | | | | | |

### Example 6. IL-2 Signaling Activity of Antibody-Cytokine/Receptor Fusions Before and After Protease Treatment

The HEK-Blue™ IL-2 reporter system expressing high affinity trimeric IL-2R (IL-2Rα, IL-2Rβ and IL-2Rγ) was used to evaluate IL-2 signaling capacity of the antibody-cytokine/receptor fusions before and after proteolytic cleavage. The cell line expresses human JAK3/STAT5 and a STAT5-inducible SEAP reporter gene. Binding of IL-2 to IL-2R leads to the secretion of SEAP that can be monitored using QUANTI-Blue™ Solution. 20 μL of serially diluted antibodies were added per well of a flat-bottom 96-well plate to which 180 μL of HEK-Blue™ IL-2 cells (approx. 100′000 cells) were added and incubated at 37° ° C. in a CO2 incubator for 24 h. 20 μL of supernatant were transferred on a flat-bottom 96-well plate containing 180 μL of QUANTI-Blue™ Solution. A control well containing noninduced HEK-Blue™ IL-2 cells was also included. After 1-3 h incubation at 37° C., SEAP levels were determined by reading the absorbance at 620-655 nm with a spectrophotometer.

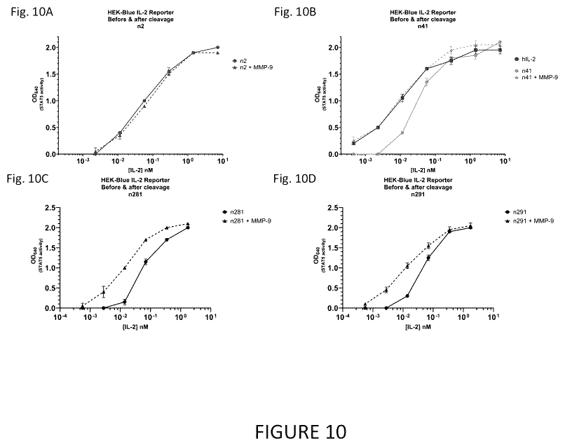

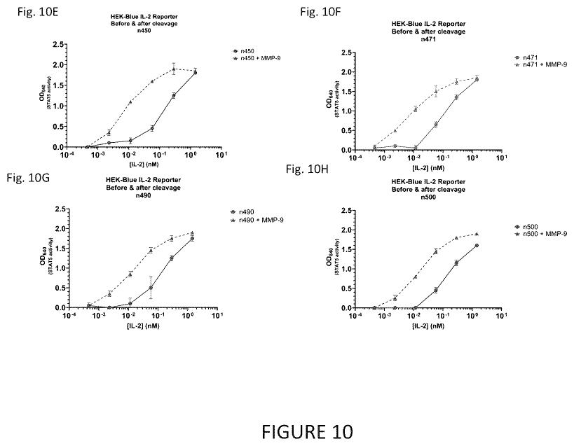

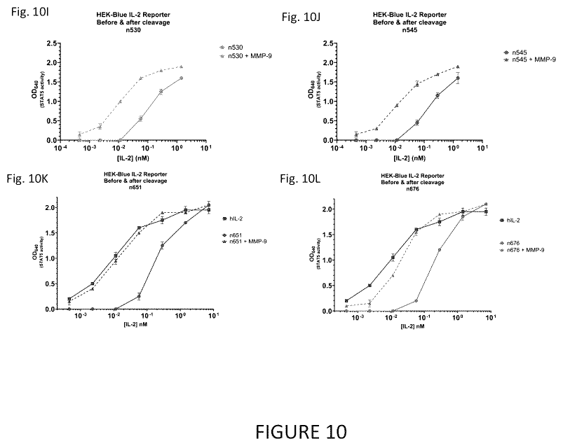

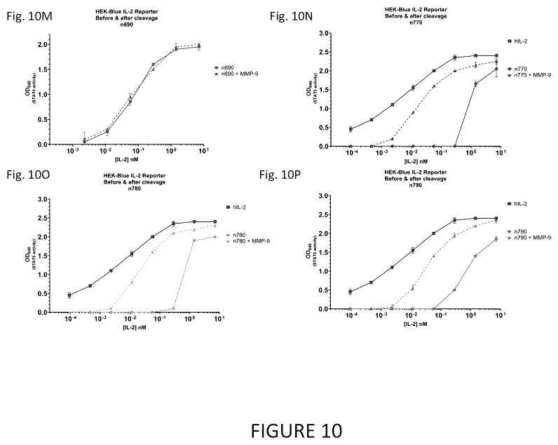

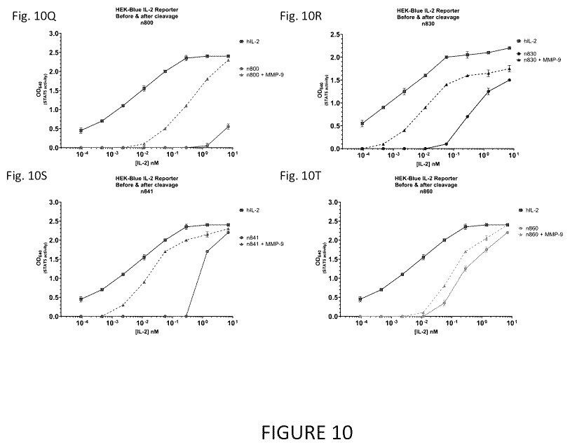

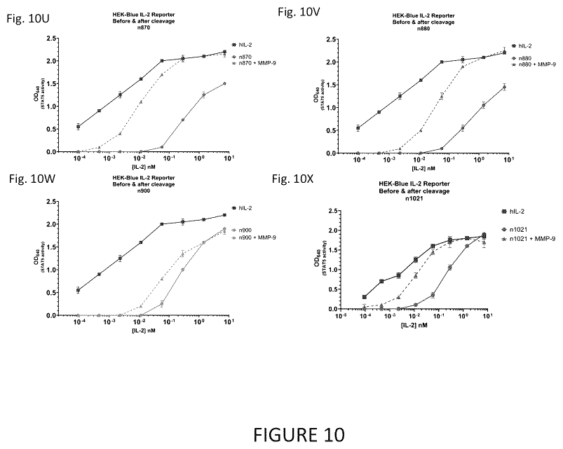

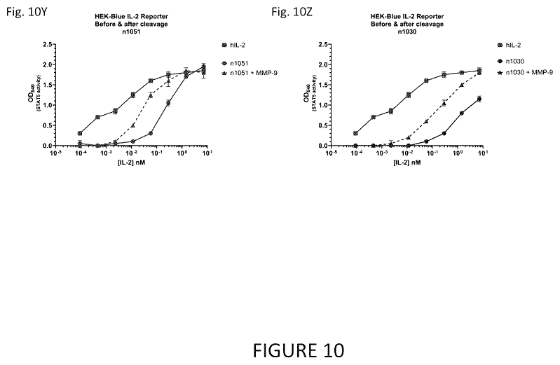

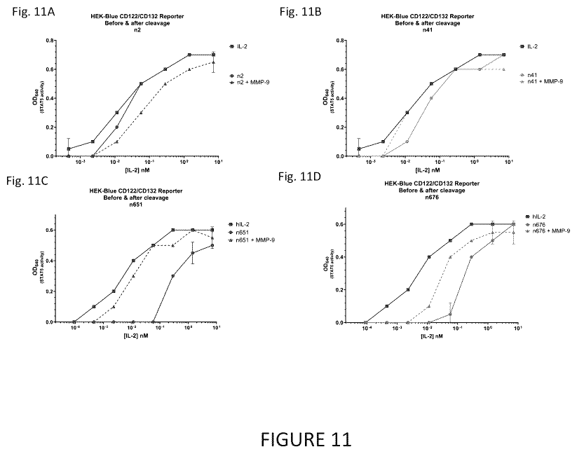

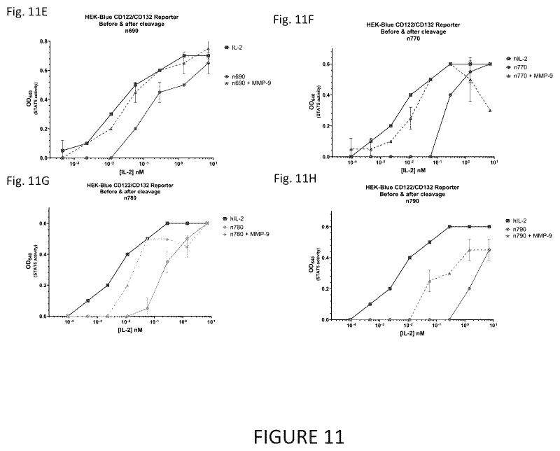

The activity measured using a dose response of antibody-cytokine/receptor fusions before and after protease treatment is shown in FIG. 10. The results summarized in Table 4 show a reduction in the IL-2 signaling activity in the non-cleaved antibody-cytokine/receptor fusions when compared to the antibody-cytokine/receptor fusions cleaved for most of the constructs listed. IL-2 mutated candidates with lower affinity to subunits of the IL-2R show a reduced signaling before and after cleavage (FIGS. 10N-10Q and 10Z). Constructs that did not contain cleavable linker (n860 and n900), showed only minimal differences before and after protease cleavage (FIGS. 10T and 10W).

TABLE 4
IL-2 activity profile of cytokine-antibody constructs before and after proteolytic cleavage.
Masking and recovery of IL-2 activity were evaluated by HEK Blue IL-2 cells (Invivogen). IL-2 activity
recovery was evaluated by dividing the IL-2 EC₅₀ obtained before and after cleavage.

IL-2 activity
(HEK Blue IL-2 αβγ reporter assay)

| | | EC50 before | EC50 after | EC50 |
|---|---|---|---|---|
| Construct | | cleavage | cleavage | ratio |
| No | Name | IL-2 (nM) | IL-2 (nM) | (BC/AC) |
| 2 | pNOVI V2K K91_IL-2 CM1_VKCK | 0.10 | 0.11 | 1 |
| 41 | pNOVI V2K K91_IL-2 CM1_VKCK_IL-2Rα_CM1_VHCH | 0.07 | 0.02 | 4 |
| 281 | pNOVI V2K K91_IL-2_CM1_VKCK_IL-2Rα WO LIN_VHCH | 0.06 | 0.01 | 4 |
| 291 | pNOVI V2K K91_IL-2_CM1_VKCK_IL-2Rα WO LIN | 0.05 | 0.01 | 4 |
| | CM1_VHCH | | | |
| 361 | pNOVI V2K K91_IL-2 WO LIN CM1 VKCK_IL-2Rα WO LIN | 0.10 | ND | — |
| | CM1_VHCH | | | |
| 450 | pNOVI V2K K91_A_IL-2_1_153_CM1 VKCK_IL- | 0.19 | 0.01 | 25 |
| | 2Rα_1_238_CM1_IL-2Rβ_27_239_CM1 VHCH | | | |
| 471 | pNOVI V2K K91_A1_IL-2_1_153_CM1 VKCK IL- | 0.12 | 0.01 | 19 |
| | 2Rα_1_238_CM1_IL-2Rβ_27_239_CM1 VHCH | | | |
| 530 | pNOVI V2K K91_A2_IL-2_1_153_CM1 VKCK IL- | 0.10 | 0.01 | 9 |
| | 2Rα_1_238_CM1_IL-2Rβ_27_239_CM1 VHCH | | | |
| 545 | pNOVI V2K K91_A3_IL-2_1_153_CM1 VKCK IL- | 0.14 | 0.01 | 10 |
| | 2Rα_1_238_CM1_IL-2Rβ_27_239_CM1 VHCH | | | |
| 490 | pNOVI V2K K91_C_IL-2_1_153_CM1 VKCK IL- | 0.16 | 0.01 | 12 |
| | 2Rα_1_185_CM1_IL-2Rβ_27_239_CM1 VHCH | | | |
| 500 | pNOVI V2K K91_D1_IL-2_1_153_CM1_IL-2Rβ_27_239_CM1 | 0.14 | 0.02 | 10 |
| | VKCK IL-2Rα_1_238_CM1_VHCH | | | |
| 651 | pNOVI V2K K91_E6_IL-2Rα_1_238_CM1_IL-2_21_153_CM1 | 0.29 | 0.02 | 18 |
| | VKCK IL-2Rβ_1_239_CM1_VHCH | | | |
| 676 | pNOVI V2K K91_E8_IL-2Rα_1_238_IL-2_21_153_CM1 VKCK | 0.28 | 0.04 | 6 |
| | IL-2Rβ_1_239_CM1_VHCH | | | |
| 690 | pNOVI V2K K91_IL-2Rα CM1_VKCK_IL-2_CM1_VHCH | 0.07 | 0.07 | 1 |
| 860 | pNOVI V2K K91_E6 NO LIN_IL-2Rα_1_238_CM1_IL- | 0.25 | 0.11 | 2 |
| | 2_21_153_CM1 VKCK IL-2Rβ_1_239_CM1_VHCH | | | |
| 900 | pNOVI V2K K91_E8 NO LIN_IL-2Rα_1_238_IL- | 0.28 | 0.08 | 3 |
| | 2_21_153_CM1 VKCK IL-2Rβ_1_239_CM1_VHCH | | | |
| 830 | pNOVI V2K K33_E6_IL-2Rα_1_238_CM1_IL-2_21_153_CM1 | 0.33 | 0.01 | 38 |
| | VKCK IL-2Rβ_1_239_CM1_VHCH | | | |
| 770 | pNOVI V2K K91_M1_IL-2Rα_1_238_CM1_IL-2 | 1.29 | 0.02 | 75 |
| | C125S_21_153_CM1 VKCK IL-2Rβ_1_239_CM1_VHCH | | | |
| 780 | pNOVI V2K K91_M2_IL-2Rα_1_238_CM1_IL-2 | 0.64 | 0.02 | 34 |
| | F42A_21_153_CM1 VKCK IL-2Rβ_1_239_CM1_VHCH | | | |
| 790 | pNOVI V2K K91_M3_IL-2Rα_1_238_CM1_IL-2 | 0.65 | 0.04 | 18 |
| | D20T_21_153_CM1 VKCK IL-2Rβ_1_239_CM1_VHCH | | | |
| 800 | pNOVI V2K K91_M4_IL-2Rα_1_238_CM1_IL-2 | 2.85 | 0.48 | 6 |
| | Q126T_21_153_CM1 VKCK IL-2Rβ_1_239_CM1_VHCH | | | |
| 841 | pNOVI V2K L1_IL-2Rβ_1_239_CM1_IL-2Rα_22_238_CM1_IL- | 1.30 | 0.02 | 73 |
| | 2_21_153_CM1 VKCK | | | |
| 870 | pNOVI V2K K91_G3_IL-2Rγ_1_262_CM1_IL-2_21_153_CM1 | 0.33 | 0.01 | 29 |
| | VKCK_IL-2Rα_1_238_CM1_VHCH | | | |
| 880 | pNOVI V2K K91_G4_IL-2Rγ_1_262_IL-2_21_153_CM1 | 0.66 | 0.04 | 17 |
| | VKCK_IL-2Rα_1_238_CM1_VHCH | | | |
| 1021 | pNOVI V2K K91_J1_IL-2Rα_1_238_CM1_IL-2_21_153_CM1 | 0.26 | 0.01 | 26 |
| | VKCK_IL-2Rα_1_238_CM1_IL-2_21_153_CM1 VHCH | | | |
| 1051 | pNOVI V2K K91_J3_IL-2Rα_1_238_IL-2_21_153_CM1 | 0.28 | 0.03 | 9 |
| | VKCK_IL-2Rα_1_238_IL-2_21_153_CM1 VHCH | | | |
| 1030 | pNOVI V2K K91_Q1_IL-2 Q126T CM1_VKCK_IL- | 0.89 | 0.23 | 4 |
| | 2Rα_CM1_VHCH | | | |

| | EC50 (nM) |
|---|---|
| hIL-2 | 0.005 +/− 0.003 |

### Example 7. IL-2 Signaling Activity of Antibody-Cytokine/Receptor Fusions Before and After Protease Treatment on Cells Expressing IL-2Rb and IL-2Rg Only

The HEK-Blue™ CD122/CD132 reporter system was used to evaluate IL-2 signaling capacity of the antibody-cytokine/receptor fusions before and after proteolytic cleavage in the context of the low-moderate affinity dimeric IL-2R (IL-2Rβ and IL-2Rγ). 20 μL of serially diluted antibodies were added per well of a flat-bottom 96-well plate to which 180 μL of HEK-Blue™ CD122/CD132 cells (approx. 100′000 cells) were added and incubated at 37° C. in a CO2 incubator for 24 h. 20 μL of supernatant were transferred on a flat-bottom 96-well plate containing 180 μL of QUANTI-Blue™ Solution. A control well containing noninduced HEK-Blue™ CD122/CD132 cells was also included. After 1-3 h incubation at 37° C., SEAP levels were determined by reading the absorbance at 620-655 nm with a spectrophotometer.

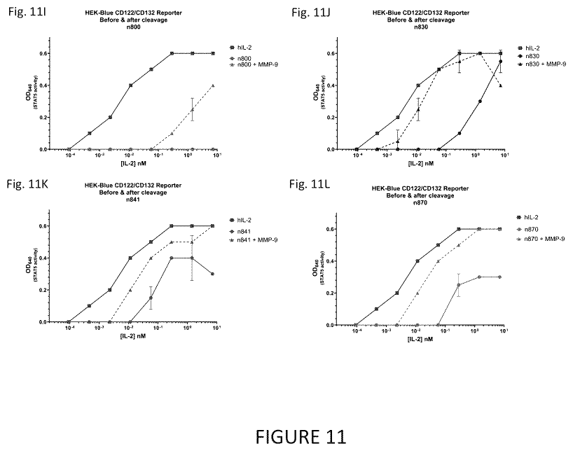

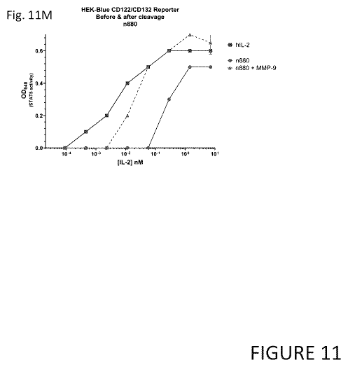

The activity measured using a dose response of antibody-cytokine/receptor fusions before and after protease treatment is shown in FIG. 11. The results summarized in Table 5 indicate that the mutation into IL-2 decreases the interaction with IL-2Rγ and reduces IL-2 signaling activity when compared to the trimeric IL-2R complex.

TABLE 5
IL-2 activity profile of cytokine-antibody constructs before and after proteolytic cleavage. Masking
and recovery of IL-2 activity were evaluated by HEK Blue CD122/CD132 cells (Invivogen). IL-2 activity
recovery was evaluated by dividing the IL-2 EC₅₀ obtained before and after cleavage.

IL-2 activity
(HEK Blue IL-2 βγ reporter assay)

| | | EC50 before | EC50 after | EC50 |
|---|---|---|---|---|
| Construct | | cleavage | cleavage | ratio |
| No | Name | IL-2 (nM) | IL-2 (nM) | (BC/AC) |
| 2 | pNOVI V2K K91_IL-2 CM1_VKCK | 0.02 | 0.09 | 0 |
| 41 | pNOVI V2K K91_IL-2 CM1_VKCK_IL-2Rα_CM1_VHCH | 0.04 | 0.01 | 3 |
| 651 | pNOVI V2K K91_E6_IL-2Rα_1_238_CM1_IL-2_21_153_CM1 | 0.24 | 0.01 | 24 |
| | VKCK IL-2Rβ_1_239_CM1_VHCH | | | |
| 676 | pNOVI V2K K91_E8_IL-2Rα_1_238_IL-2_21_153_CM1 | 0.18 | 0.03 | 6 |
| | VKCK IL-2Rβ_1_239_CM1_VHCH | | | |
| 690 | pNOVI V2K K91_IL-2Rα CM1_VKCK_IL-2_CM1_VHCH | 0.12 | 0.03 | 4 |
| 830 | pNOVI V2K K33_E6_IL-2Rα_1_238_CM1_IL-2_21_153_CM1 | 1.74 | 0.01 | 147 |
| | VKCK IL-2Rβ_1_239_CM1_VHCH | | | |
| 770 | pNOVI V2K K91_M1_IL-2Rα_1_238_CM1_IL-2 | 0.22 | 0.01 | 19 |
| | C125S_21_153_CM1 VKCK IL-2Rβ_1_239_CM1_VHCH | | | |
| 780 | pNOVI V2K K91_M2_IL-2Rα_1_238_CM1_IL-2 | 0.23 | 0.01 | 17 |
| | F42A_21_153_CM1 VKCK IL-2Rβ_1_239_CM1_VHCH | | | |
| 790 | pNOVI V2K K91_M3_IL-2Rα_1_238_CM1_IL-2 | 1.44 | 0.07 | 19 |
| | D20T_21_153_CM1 VKCK IL-2Rβ_1_239_CM1_VHCH | | | |
| 800 | pNOVI V2K K91_M4_IL-2Rα_1_238_CM1_IL-2 | No IL-2 | 1.14 | — |
| | Q126T_21_153_CM1 VKCK IL-2Rβ_1_239_CM1_VHCH | activity | | |
| | | detected | | |
| 841 | pNOVI V2K L1_IL-2Rβ_1_239_CM1_IL- | 0.06 | 0.02 | 3 |
| | 2Rα_22_238_CM1_IL-2_21_153_CM1 VKCK | | | |
| 870 | pNOVI V2K K91_G3_IL-2Rγ_1_262_CM1_IL-2_21_153_CM1 | 0.26 | 0.03 | 10 |
| | VKCK_IL-2Rα_1_238_CM1_VHCH | | | |
| 880 | pNOVI V2K K91_G4_IL-2Rγ_1_262_IL-2_21_153_CM1 | 0.28 | 0.02 | 12 |
| | VKCK_IL-2Rα_1_238_CM1_VHCH | | | |

| | EC50 (nM) |
|---|---|
| hIL-2 | 0.01 +/− 0.007 |

These examples show that in the antibody-cytokine/receptor fusion both the antibody binding and the cytokine activity are reciprocally impaired. Proteolytic cleavage, and release of the antibody and cytokine can restore a full binding and signaling activity.

### Example 8. Sequences Used to Design Masking Domains

| | |
|---|---|
| IL-2 | MYRMQLLSCIALSLALVTNSAPTSSSTKKTQLQLEHLLLDLQMILNGINNY |
| | KNPKLTRMLTFKFYMPKKATELKHLQCLEEELKPLEEVLNLAQSKNFH |
| | LRPRDLISNINVIVLELKGSETTFMCEYADETATIVEFLNRWITFCQSIIST |
| | LT (SEQ ID: 1) |
| IL-2Rα | MDSYLLMWGLLTFIMVPGCQAELCDDDPPEIPHATFKAMAYKEGTMLN |
| | CECKRGFRRIKSGSLYMLCTGNSSHSSWDNQCQCTSSATRNTTKQVTP |
| | QPEEQKERKTTEMQSPMQPVDQASLPGHCREPPPWENEATERIYHFVV |
| | GQMVYYQCVQGYRALHRGPAESVCKMTHGKTRWTQPQLICTGEMET |
| | SQFPGEEKPQASPEGRPESETSCLVTTTDFQIQTEMAATMETSIFTTE |
| | (SEQ ID: 2) |
| IL-2Rβ | MAAPALSWRLPLLILLLPLATSWASAAVNGTSQFTCFYNSRANISCVWSQD |
| | GALQDTSCQVHAWPDRRRWNQTCELLPVSQASWACNLILGAPDSQKL |
| | TTVDIVTLRVLCREGVRWRVMAIQDFKPFENLRLMAPISLQVVHVETH |
| | RCNISWEISQASHYFERHLEFEARTLSPGHTWEEAPLLTLKQKQEWICL |
| | ETLTPDTQYEFQVRVKPLQGEFTTWSPWSQPLAFRTKPAALGKD (SEQ |
| | ID: 3) |
| IL-2Rγ | MLKPSLPFTSLLFLQLPLLGVGLNTTILTPNGNEDTTADFFLTTMPTDSLS |
| | VSTLPLPEVQCFVFNVEYMNCTWNSSSEPQPTNLTLHYWYKNSDNDK |
| | VQKCSHYLFSEEITSGCQLQKKEIHLYQTFVVQLQDPREPRRQATQMLK |
| | LQNLVIPWAPENLTLHKLSESQLELNWNNRFLNHCLEHLVQYRTDWD |
| | HSWTEQSVDYRHKFSLPSVDGQKRYTFRVRSRFNPLCGSAQHWSEWS |
| | HPIHWGSNTSKENPFLFALEA (SEQ ID: 4) |
| IL-15 | MRISKPHLRSISIQCYLCLLLNSHFLTEAGIHVFILGCFSAGLPKTEANWVNV |
| | ISDLKKIEDLIQSMHIDATLYTESDVHPSCKVTAMKCFLLELQVISLESG |
| | DASIHDTVENLIILANNSLSSNGNVTESGCKECEELEEKNIKEFLQSFVHI |
| | VQMFINTS(SEQ ID: 5) |
| IL-15Rα | MAPRRARGCRTLGLPALLLLLLLRPPATRGITCPPPMSVEHADIWVKSYSL |
| Sushi 1 | YSRERYICNSGFKRKAGTSSLTECVLNKATNVAHWTTPSLKCIRD (SEQ |
| | ID: 6) |
| CM1 | VHMPLGFLGP (SEQ ID: 7) |
| CM2 | TSTSGRSANPRG (SEQ ID: 8) |
| CM3 | EAGRSANHTPAGLTGP (SEQ ID: 9) |
| K91 and | EVQLLESGGGLVQPGGSLRLSCAASGFTFSSYAMSWVRQAPGKGLEW |
| K33 | VSAISGSGGSTYYADSVKGRFTISRDNSKNTLYLQMNSLRAEDTAVYY |
| heavy | CAKSYGAFDYWGQGTLVTVSSASTKGPSVFPLAPSSKSTSGGTAALGC |
| chain | LVKDYFPEPVTVSWNSGALTSGVHTFPAVLQSSGLYSLSSVVTVPSSSL |
| variable | GTQTYICNVNHKPSNTKVDKRVEPKSCDKTHTCPPCPAPELLGGPSVFL |
| region | FPPKPKDTLMISRTPEVTCVVVDVSHEDPEVKFNWYVDGVEVHNAKT |
| and | KPREEQYNSTYRVVSVLTVLHQDWLNGKEYKCKVSNKALPAPIEKTIS |
| common | KAKGQPREPQVYTLPPSREEMTKNQVSLTCLVKGFYPSDIAVEWESNG |
| region | QPENNYKTTPPVLDSDGSFFLYSKLTVDKSRWQQGNVFSCSVMHEALH |
| | NHYTQKSLSLSPG (SEQ ID: 16) |
| K91 | DIQMTQSPSSLSASVGDRVTITCNVDQVIANWLNWYQQKPGKAPKLLI |
| kappa | YAASSLQSGVPSRFSGSGSGTDFTLTISSLQPEDFATYYCQQMHPRAPK |
| light | QFGQGTKVEIKRTVAAPSVFIFPPSDEQLKSGTASVVCLLNNFYPREAK |
| chain | VQWKVDNALQSGNSQESVTEQDSKDSTYSLSSTLTLSKADYEKHKVY |
| | ACEVTHQGLSSPVTKSFNRGEC (SEQ ID: 17) |
| K33 | DIQMTQSPSSLSASVGDRVTITCRASQSISSYLNWYQQKPGKAPKLLIYA |
| kappa | ASSLQSGVPSRFSGSGSGTDFTLTISSLQPEDFATYYCQQFHKRSPQKFG |
| light | QGTKVEIKRTVAAPSVFIFPPSDEQLKSGTASVVCLLNNFYPREAKVQW |
| chain | KVDNALQSGNSQESVTEQDSKDSTYSLSSTLTLSKADYEKHKVYACEV |
| | THQGLSSPVTKSFNRGEC (SEQ ID: 18) |

Sequence in italics denote leader sequence

### REFERENCES

Labrijn A F, Janmaat M L, Reichert J M, Parren PWHI. Bispecific antibodies: a mechanistic review of the pipeline. Nat Rev Drug Discov. 2019 August; 18(8):585-608.
Hansel T T, Kropshofer H, Singer T, Mitchell J A, George A J T. The safety and side effects of monoclonal antibodies. Nat Rev Drug Discov. 2010 April; 9(4):325-38.
Lucchi R, Bentanachs J, Oller-Salvia B. The Masking Game: Design of Activatable Antibodies and Mimetics for Selective Therapeutics and Cell Control. ACS.
Bleuez C, Koch W F, Urbach C, Hollfelder F, Jermutus L. Exploiting protease activation for therapy. Drug Discov Today. 2022 June; 27(6): 1743-54.
Chen I J, Chuang C H, Hsieh Y C, Lu Y C, Lin W W, Huang C C, et al. Selective antibody activation through protease-activated pro-antibodies that mask binding sites with inhibitory domains. Sci Rep. 2017 Sep. 14; 7:11587.
Vasiljeva O, Hostetter D R, Moore S J, Winter M B. The multifaceted roles of tumor-associated proteases and harnessing their activity for prodrug activation. Biol Chem. 2019
Mahmood N, Mihalcioiu C, Rabbani S A. Multifaceted Role of the Urokinase-Type Plasminogen Activator (uPA) and Its Receptor (uPAR): Diagnostic, Prognostic, and Therapeutic Applications. Front Oncol. 2018 Feb. 12; 8:24.
Berraondo P, Sanmamed M F, Ochoa M C, Etxeberria I, Aznar M A, Pérez-Gracia J L, et al. Cytokines in clinical cancer immunotherapy. Br J Cancer. 2019 January; 120(1):6-15.
Puskas J, Skrombolas D, Sedlacek A, Lord E, Sullivan M, Frelinger J. Development of an attenuated interleukin-2 fusion protein that can be activated by tumour-expressed proteases. Immunology. 2011 June; 133(2):206-20.

1. An antibody fusion protein having the following structure:
(a) a first antigen binding domain comprising a first heavy chain polypeptide (H1) comprising) and a first light chain polypeptide (L1); and
(b) a second antigen binding domain comprising a second heavy chain polypeptide (H2) and a second light chain polypeptide (L2);
wherein a cytokine is linked via a first protease cleavable linker:
(i) to an N-terminus of the L1 and/or the L2;
(ii) to the N-terminus of the H1 and/or H2; or
(iii) to an N-terminus of the L1, L2, H1 and/or H2; and
wherein the first antigen binding domain and the second antigen binding domain are not specific for the cytokine.

2. The antibody fusion protein of claim 1, further comprising a second protease linker.

3. The antibody fusion protein of claim 2, further comprising at least a first portion of the cognate receptor of the cytokine linked via the second protease linker.

4. The antibody fusion protein of claim 3, wherein the second protease linker is linked to the N-terminus of the H1 and/or H2.

5. The antibody fusion protein of claim 3, wherein the second protease linker is linked to the N-terminus of the L1 and/or L2.

6. The antibody fusion protein of claim 3, wherein the second protease linker is linked to the cytokine.

7. The antibody fusion protein of claim 3, further comprising at least a second portion of the cognate receptor of the cytokine.

8. The antibody fusion protein of claim 7, further comprising a third protease linker.

9. The antibody fusion protein of claim 8, wherein the at least a second portion of the cognate receptor of the cytokine is linked to the antibody fusion via a third protease linker.

10. The antibody fusion of claim 9, wherein the third protease linker is linked to any one of the N terminus of H1, H2, L1, L2, the first portion of the cognate receptor of the cytokine, or the cytokine.

11. The antibody fusion protein of claim 10, further comprising a non-cleavable linker.

12. The antibody fusion protein of claim 1, wherein the cytokine is IL-2, IL-15, a mutated IL-2, or a mutated IL-15.

13. The antibody fusion protein of claim 3, wherein the at least a first portion of the cognate receptor is any one of the extracellular portions of IL-2Rα, IL-2RB, IL-2Rγ, IL-15Rα Sushi 1.

14. The antibody fusion protein of claim 7, wherein the at least a second portion of the cognate receptor is any one of the extracellular portions of IL-2Rα, IL-2Rβ, IL-2Rγ, IL-15Rα Sushi 1.

15. The antibody fusion protein of claim 1, wherein the antibody fusion protein is specific for CD47.

16. The antibody fusion protein of claim 15, wherein the antibody protein fusion protein comprises the K91 or K33 antibody.

17. The antibody fusion protein of claim 12, wherein the IL-2 cytokine comprises one or more of a C125S mutation, a F42A mutation, a D20T mutation, or a Q126T mutation.

18. The antibody fusion protein of claim 1, wherein the antibody fusion protein further comprises a modified Fc domain.

19. The antibody fusion protein of claim 18, wherein the antibody fusion protein is a human IgG1, or a human IgG2, or a human IgG3, or a human IgG4, or a human IgA, or a human IgE, or a human IgM.

20. The antibody fusion protein of claim 1, wherein the antibody fusion protein is a bispecific antibody, wherein the first antigen binding domain binds to a first antigen and the second antigen binding domain binds to a second antigen, wherein the first antigen and the second antigen are not the same antigen.

21. A method of masking the binding activity of an antibody fusion protein of claim 1, wherein the cognate target binding activity of the antibody fusion protein is reduced.

22. The method of masking of claim 21, wherein the cognate target binding activity of the antibody fusion protein is reduced by steric hindrance of the first antigen binding domain and/or the second antigen binding domain.

23. The method of masking of claim 22, wherein the steric hindrance is removed by the cleaving activity of a matrix metalloproteinase.

24. The method of masking of any one of claims 21-23, wherein the antibody fusion protein binding activity before and after cleavage by a matrix metalloproteinase is determined by surface plasmon resonance, or by bio-layer interferometry.

25. The method of masking of claim 24, wherein the binding activity of the antibody protein fusion is determined before cleavage (BC) and after cleavage (AC), wherein the binding recovery is determined by a ratio of BC to AC.

26. The method of masking of claim 25, wherein the ratio of BC to AC is at least 5, or at least 10, or at least 20, or at least 30, or at least 40, or at least 50, or at least 60, or at least 70, or at least 80, or at least 90, or at least 100, or at least 150.

27. The method of masking of any one of claims 21-23, wherein the cytokine activity before and after cleavage by a matrix metalloproteinase is determined by measuring the cytokine activity using a cytokine signaling cell reporter system.

28. The method of masking of claim 27, wherein the EC50 cytokine signaling activity of the antibody protein fusion is determined before cleavage (BC) EC50 and after cleavage (AC) EC50, wherein the recovery of cytokine signaling activity is determined by a ratio of BC to AC.

29. The method of masking of claim 28, wherein the ratio of BC to AC is at least 5, or at least 10, or at least 20, or at least 30, or at least 40, or at least 50, or at least 60, or at least 70, or at least 80, or at least 90, or at least 100, or at least 120.

30. The method of treating a human disease in a subject by administering a therapeutically effective amount of an antibody fusion protein of claim 1.

31. The method of treating a human disease of claim 30, wherein the antibody fusion protein is activated by a matrix metalloproteinases cleavage within or near tumor tissue.

32. The method of treating a human disease of any one of claim 30 or 31, wherein the human disease is cancer.

33. The method of treating human disease of claim 32, wherein the cancer is one of bladder cancer, breast cancer, colon and rectal cancer, lung cancer, melanoma cancer, endometrial cancer, kidney cancer, leukemia, lymphoma, pancreatic cancer, prostate cancer, brain cancer, central nervous system cancer, gastric cancer, esophageal cancer, thyroid cancer, head and neck cancer, ovarian cancer, or oral cancer.
